(19) United States
(12) Patent Application Publication
(10) Pub. No.: US 2025/0312344 A1
(43) Pub. Date: Oct. 9, 2025

(54) COMBINATION THERAPIES USING PRMT5 INHIBITORS AND IMMUNE CHECKPOINT INHIBITORS FOR THE TREATMENT OF CANCER

(71) Applicant: MIRATI THERAPEUTICS, INC., PRINCETON, NJ (US)

(72) Inventors: LARS DANIEL ENGSTROM, ESCONDIDO, CA (US); PETER OLSON, SAN DIEGO, CA (US); JILL HALLIN, SAN DIEGO, CA (US)

(73) Assignee: MIRATI THERAPEUTICS, INC., PRINCETON, NJ (US)

(21) Appl. No.: 19/171,411

(22) Filed: Apr. 7, 2025

Related U.S. Application Data
(60) Provisional application No. 63/631,028, filed on Apr. 8, 2024.

(51) Int. Cl. A61K 31/502; A61K 39/00; A61K 39/395; A61P 35/00

(52) U.S. Cl. CPC A61K 31/502; A61K 39/3955; A61P 35/00; A61K 2039/507

(57) ABSTRACT

This disclosure relates to methods of treating cancer. This disclosure further relates to treating cancer in a subject with compounds that are methylthioadenosine (MTA)-cooperative PRMT5 inhibitors, particularly in combination with one or more immuno-oncology checkpoint inhibitors.

### CROSS REFERENCE TO RELATED APPLICATIONS

This application claims the benefit of U.S. Provisional Application No. 63/631,028, filed Apr. 8, 2024, the entire content of which is hereby incorporated herein by reference.

### BACKGROUND OF THE DISCLOSURE

### Field of the Disclosure

This disclosure relates to methods of treating cancer. This disclosure further relates to treating cancer in a subject with compounds that are inhibitors of protein arginine N-methyl transferase 5 (PRMT5), particularly in combination with immuno-oncology checkpoint inhibitors.

### Description of Related Art

PRMT5 is a type II arginine methyltransferase that catalyzes the transfer of a methyl group from S-adenosyl-L-methionine (SAM) to an omega-nitrogen of the guanidino function of protein L-arginine residues (omega-monomethylation) and the transfer of a second methyl group to the other omega-nitrogen, yielding symmetric dimethylarginine (sDMA). PRMT5 forms a complex with methylosome protein 50 (MEP50), which is required for substrate recognition and orientation and is also required for PRMT5-catalyzed histone 2A and histone 4 methyltransferase activity (e.g., see Ho et al. (2013) PLOS ONE 8 (2): e57008).

Homozygous deletions of p16/CDKN2a are prevalent in cancer and these mutations commonly involve the co-deletion of adjacent genes, including the gene encoding methylthioadenosine phosphorylase (MTAP). It is estimated that approximately 15% of all human cancers have a homozygous deletion of the MTAP gene (e.g., see Firestone & Schramm (2017) J. Am. Chem Soc. 139 (39): 13754-13760).

Cells lacking MTAP activity have elevated levels of the MTAP substrate, methylthioadenosine (MTA), which is a potent inhibitor of PRMT5. Inhibition of PRMT5 activity results in reduced methylation activity and increased sensitivity of cellular proliferation to PRMT5 depletion or loss of activity. Hence, the loss of MTAP activity reduces methylation activity of PRMT5 making the cells selectively dependent on PRMT5 activity.

As with individual cancer cells developing resistance to anticancer therapies, a large portion of patients also acquire resistance to immune checkpoint blockade (ICB) agents. To overcome this resistance, monoclonal antibodies that target either PD-1 or PD-L1 can boost the immune response against cancer cells. These antibodies have been found to be useful in treating skin cancer, non-small cell lung cancer (NSCLC), kidney cancer, bladder cancer, head and neck cancers, and Hodgkin lymphoma. However, later relapses are also emerging in patient with prior clinical benefit with the PD-1 or PD-L1 antibody, suggesting emerging resistance. (Jenkins R W, Barbie D A, and Flaherty K T. Mechanism of resistance to immune checkpoint inhibitors. BR J Cancer. 2018; 118 (1): 9-16).

A role for PRMT5 in T regulatory cells (Tregs) and CTLA-4 has been demonstrated in preclinical models (See Nagai, Y., PRMT5 Associates With the FOXP3 Homomer and When Disabled Enhances Targeted p185erbB2/neu Tumor Immunotherapy; Front Immunol, 2019). PRMT5 deletion in Treg cells resulted in dysfunctional Tregs and autoimmunity in mice. Furthermore, Treg cells from PRMT5 knock out mice exhibited reduced CTLA4 expression. PRMT5 has been mechanistically linked to immune suppression and inhibition of PRMT5 has been posited as a strategy to increase the responsiveness of cancers to immune check point inhibitor (Abe, Y., The Role of PRMT5 in Ommuno-Oncology; Genes (Basel), 2023). For all the foregoing reasons, there is a need to develop combination therapies using PRTM5 inhibitors and ICB therapy, including anti-PD-1 and anti-PD-L1 inhibitors, for treating a wide range of cancers.

### SUMMARY OF THE DISCLOSURE

One aspect of the disclosure provides methods for treating cancer in a subject. Such methods include administering to the subject a therapeutically effective amount of an immuno-oncology checkpoint inhibitor and a therapeutically effective amount of a PRMT5 inhibitor.

Also provided herein is a method for treating cancer in a subject in need thereof. Such methods include determining that the cancer is associated with MTAP homozygous deletion (e.g., an MTAP-associated cancer). Such methods further include administering to the subject a therapeutically effective amount of one or more immuno-oncology checkpoint inhibitor and a therapeutically effective amount of a PRMT5 inhibitor.

These and other features and advantages of the present invention will be more fully understood from the following detailed description taken together with the accompanying claims. It is noted that the scope of the claims is defined by the recitations therein and not by the specific discussion of features and advantages set forth in the present description.

### DETAILED DESCRIPTION OF THE DISCLOSURE

Before the disclosed processes and materials are described, it is to be understood that the aspects described herein are not limited to specific embodiments, and as such can, of course, vary. It is also to be understood that the terminology used herein is for the purpose of describing particular aspects only and, unless specifically defined herein, is not intended to be limiting.

As describe above, both MTAP^{del} and KRAS mutations are prevalent in many cancers, and that resistance to immune checkpoint blockade (ICB) therapy can occur with oncogenic KRAS mutations. The present inventors have advantageously found a combination therapy to target cancers with both of these characteristics. In one aspect, the present disclosure provides a method for treating cancer in a subject, the method includes administering to the subject a therapeutically effective amount of one or more immuno-oncology checkpoint inhibitor and a therapeutically effective amount of a protein arginine N-methyl transferase 5 (PRMT5) inhibitor, wherein the PRMT5 inhibitor is methylthioadenosine (MTA)-cooperative PRMT5 inhibitor.

### Combination Therapy

In view of the present disclosure, the methods and compositions described herein can be configured by the person of ordinary skill in the art to meet the desired need. The present disclosure provides improvements in treating cancer in a subject. As used herein, the terms “subject” or “patient” are used interchangeably, refers to any animal, including mammals, and most preferably humans.

The methods provided herein may be used for the treatment of a wide variety of cancer including tumors such as lung, prostate, breast, brain, skin, cervical carcinomas, testicular carcinomas, etc. More particularly, cancers that may be treated by the compositions and methods of the invention include, but are not limited to tumor types such as astrocytic, breast, cervical, colorectal, endometrial, esophageal, gastric, head and neck, hepatocellular, laryngeal, lung, oral, ovarian, prostate and thyroid carcinomas and sarcomas. More specifically, these compounds can be used to treat: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma.

In certain embodiments of the methods of the disclosure, the cancer is a MTAP-associated cancer. For example, in certain embodiments, the cancer comprises MTAP gene homozygous deletion (MTAP^{DEL}). The subject may be identified or diagnosed as having MTAP-associated cancer where, for example, MTAP^{DEL} is determined using a suitable assay or a kit. Alternatively, the subject is suspected of having MTAP-associated cancer or the subject has a clinical record indicating that the subject has MTAP-associated cancer.

In certain embodiments of the methods of the disclosure, the cancer comprises a KRAS^{G12C} gene mutation. The subject may be identified or diagnosed as having KRAS^{G12C} cancer where KRAS^{G12C} mutation is determined using a suitable assay or a kit. Alternatively, the subject is suspected of having the KRAS^{G12C} cancer or the subject has a clinical record indicating that the subject has the KRAS^{G12C} cancer.

In certain embodiments of the methods of the disclosure, the cancer may further comprise a cyclin-dependent kinase inhibitor 2A (CDKN2A) gene homozygous deletion (CDKN2A^{DEL}). The subject may be identified or diagnosed as having CDKN2A^{DEL} where the deletion is determined using a suitable assay or a kit. Alternatively, the subject is suspected of having the CDKN2A^{DEL} cancer, or the subject has a clinical record indicating that the subject has the CDKN2A^{DEL} cancer.

In some embodiments of any of the methods or uses described herein, an assay is used to determine whether the patient has MTAP^{DEL} and/or KRAS^{G12C} and/or CDKN2A^{DEL} using a sample (e.g., a biological sample or a biopsy sample such as a paraffin-embedded biopsy sample) from a subject. Such assay includes, but is not limited to, next generation sequencing, immunohistochemistry, fluorescence microscopy, break apart FISFI analysis, Southern blotting. Western blotting, FACS analysis, Northern blotting, and PCR-based amplification (e.g., RT-PCR and quantitative real-time RT-PCR). As is well known in the art, the assays are typically performed, e.g., with at least one labelled nucleic acid probe or at least one labelled antibody or antigen-binding fragment thereof.

In certain embodiments, the cancer in the methods of the disclosure is selected from lung cancer, pancreatic cancer, colon cancer, head and neck cancer, bladder cancer, esophageal cancer, lymphoma, stomach cancer, skin cancer, breast cancer, and brain cancer.

In certain embodiments, the cancer in the methods of the disclosure is selected from lung cancer, pancreatic cancer, colon cancer, head and neck cancer, esophageal cancer, and melanoma.

In certain embodiments, the cancer in the methods of the disclosure is selected from lung cancer (e.g., mesothelioma or non-small cell lung cancer (NSCLC) including adenocarcinoma and squamous cell), pancreatic cancer, colon cancer, head and neck cancer (such as squamous cell carcinoma (HNSCC)), bladder cancer, esophageal cancer, lymphoma (e.g., diffuse large B-cell lymphoma), stomach cancer, melanoma, breast cancer, and brain cancer (e.g., glioblastoma multiforme and glioma).

In certain embodiments, the cancer in the methods of the disclosure is selected from lung cancer (e.g., mesothelioma or NSCLC, including adenocarcinoma and squamous cell), pancreatic cancer, colon cancer, head and neck cancer (e.g. squamous cell carcinoma (HNSCC)), esophageal cancer, and melanoma.

In certain embodiments, the cancer in the methods of the disclosure is selected from mesothelioma, NSCLC (e.g., adenocarcinoma and squamous cell), pancreatic cancer, HNSCC, and colon cancer.

In one embodiment of the methods of the disclosure, the cancer is lung cancer. For example, the lung cancer may be NSCLC (e.g., adenocarcinoma and squamous cell) or mesothelioma.

In certain embodiment, the cancer is NSCLC.

In one embodiment of the methods of the disclosure, the cancer is pancreatic cancer.

In one embodiment of the methods of the disclosure, the cancer is colon cancer.

The PRMT5 inhibitor of the disclosure and/or the immuno-oncology checkpoint inhibitor of the disclosure may be provided as a pharmaceutical composition comprising a therapeutically effective amount of such inhibitor and a pharmaceutically acceptable carrier, excipient, and/or diluents. The PRMT5 inhibitor of the disclosure and/or the immuno-oncology checkpoint inhibitor of the disclosure may be formulated by any method well known in the art and may be prepared for administration by any route, including, without limitation, parenteral, oral, sublingual, transdermal, topical, intranasal, intratracheal, or intrarectal. In certain embodiments, the PRMT5 inhibitor of the disclosure and/or the immuno-oncology checkpoint inhibitor of the disclosure are administered intravenously in a hospital setting. In certain other embodiments, administration may preferably be by the oral route.

The characteristics of the carrier will depend on the route of administration. As used herein, the term “pharmaceutically acceptable” means a non-toxic material that is compatible with a biological system such as a cell, cell culture, tissue, or organism, and that does not interfere with the effectiveness of the biological activity of the active ingredient(s). Thus, pharmaceutical compositions of the disclosure may contain, in addition to the inhibitor, diluents, fillers, salts, buffers, stabilizers, solubilizers, and other materials well known in the art. The preparation of pharmaceutically acceptable formulations is described in, e.g., Remington's Pharmaceutical Sciences, 18^{th} Edition, ed. A. Gennaro, Mack Publishing Co., Easton, Pa., 1990.

The PRMT5 inhibitor and the immuno-oncology checkpoint inhibitor of the disclosure are administered in a therapeutically effective amount. As used herein, the phrase “therapeutically effective amount” or “effective amount” refers to the amount of active agent that elicits the biological or medicinal response that is being sought in a tissue, system, subject or human by a researcher, medical doctor or other clinician. In general, the therapeutically effective amount is sufficient to deliver the biological or medicinal response to the subject without causing serious toxic effects. A dose of the active agent may be in the range from about 0.01 to 300 mg/kg per day, such as 0.1 to 100 mg/kg per day, more generally 0.5 to about 25 mg/kg body weight of the recipient per day. A typical topical dosage will range from 0.01 to 3% wt/wt in a suitable carrier.

In certain embodiments of the methods of the disclosure, the therapeutically effective amount of the PRMT5 inhibitor is in the range of about 0.01 to 300 mg/kg per day. For example, in certain embodiments, the therapeutically effective amount of the PRMT5 inhibitor is in the range of about 0.1 to 100 mg/kg per day, or 25 to 100 mg/kg per day, or 50 to 100 mg/kg per day.

In certain embodiments, the therapeutically effective amount of the PRMT5 inhibitor is less than 1% of, e.g., less than 10%, or less than 25%, or less than 50% of the clinically-established therapeutic amount (e.g., such as the amount required when the PRMT5 inhibitor is administered by itself).

In certain embodiments, the therapeutically effective amount of the PRMT5 inhibitor is administered once daily.

In certain embodiments of the methods of the disclosure, the therapeutically effective amount of the immuno-oncology checkpoint inhibitor is in the range of about 0.01 to 300 mg/kg per day. For example, in certain embodiments, the therapeutically effective amount of the immuno-oncology checkpoint inhibitor is in the range of about 0.1 to 100 mg/kg per day, or 0.1 to 50 mg/kg per day, or 10 to 100 mg/kg per day, or 10 to 50 mg/kg per day.

In certain embodiments, the therapeutically effective amount of the immuno-oncology checkpoint inhibitor is less than 1% of, e.g., less than 10%, or less than 25%, or less than 50% of the clinically-established therapeutic amount (e.g., such as the amount required when the immuno-oncology checkpoint inhibitor is administered by itself).

Combination therapy, in defining use of PRMT5 inhibitor and the immuno-oncology checkpoint inhibitor of the present disclosure, is intended to embrace administration of each agent in a sequential manner in a regimen that will provide beneficial effects of the drug combination (e.g., the PRMT5 inhibitor and the immuno-oncology checkpoint inhibitor of the disclosure can be formulated as separate compositions that are given sequentially), and is intended as well to embrace co-administration of these agents in a substantially simultaneous manner, such as in a single dosage form having a fixed ratio of these active agents or in multiple or a separate dosage forms for each agent. The disclosure is not limited in the sequence of administration: the PRMT5 inhibitor of the disclosure may be administered either prior to or after (i.e., sequentially), or at the same time (i.e., simultaneously) as administration of the immuno-oncology checkpoint inhibitor of the disclosure.

The methods of disclosure are useful as a first-line treatment. Thus, in certain embodiments of the methods of the disclosure, the subject has not previously received another first-line of therapy.

The methods of disclosure are also useful as a first-line maintenance or a second-line treatment. Thus, in certain embodiments of the methods of the disclosure, the subject has previously completed another first-line of therapy. For example, the methods of the disclosure, in certain embodiments, may provide a delay in progression and relapse of cancer in subjects that have previously completed another first-line chemotherapy. For example, in certain embodiments, the subject has previously completed a platinum- and/or taxane-based chemotherapy (e.g., carboplatin, cisplatin, oxaliplatin, paclitaxel, docetaxel, and the like). In certain embodiments of the methods of the disclosure, the subject has previously completed another first-line chemotherapy and is in partial response to such chemotherapy.

### Immuno-Oncology Checkpoint Inhibitors

As described above, the methods of the disclosure include administering one or more immune-oncology checkpoint inhibitors.

In various embodiments of the methods of the disclosure, the immuno-oncology checkpoint inhibitor is selected from a CTLA-4 inhibitor, a PD-1 inhibitor, a PD-L1 inhibitor, or combinations thereof.

For example, in some embodiments as described herein, the CTLA-4 inhibitor is selected from ipilimumab, tremelimumab, and combinations thereof. In some embodiments as described herein, the CTLA-4 inhibitor is selected from ipilimumab. In some embodiments, the CTLA-4 inhibitor is selected from termeliumumab.

As used herein, Programmed cell death protein 1 (PD-1) is a 55 kDa type I transmembrane protein that is part of the Ig gene superfamily that delivers negative cellular signals upon interaction with its two ligands, PD-L1 or PD-L2, to suppress the immune response.

As used herein, a “PD-1/PD-L1 inhibitor” refers to an agent that is capable of negatively modulating or inhibiting all or a portion of the PD-1/PD-L1 axis signaling activity and include agents that block PD-1 or PD-L1 Examples include PD-1 and PD-L1 binding antagonists such as anti-PD-1 antibodies, antigen binding fragments thereof, immunoadhesins, aptamers, fusion proteins, and oligopeptides. In some embodiments, the PD-1 binding antagonist is an anti-PD-1 antibody. In some embodiments, the PD-L1 binding antagonist is an anti-PD-L1 antibody.

The term “PD-1 binding antagonist” as used herein refers to a PD-1 inhibitor, i.e., a molecule that decreases, blocks, inhibits, abrogates or interferes with signal transduction resulting from the interaction of PD-1 with one or more of its binding partners, such as PD-L1 and/or PD-L2. In some embodiments, the PD-1 inhibitor is a molecule that inhibits the binding of PD-1 to its binding partners. In a specific aspect, the PD-1 inhibitor inhibits the binding of PD-1 to PD-L1 and/or PD-L2. For example, PD-1 inhibitors include anti-PD-1 antibodies, antigen binding fragments thereof, immunoadhesins, fusion proteins, oligopeptides and other molecules that decrease, block, inhibit, abrogate or interfere with signal transduction resulting from the interaction of PD-1 with PD-L1 and/or PD-L2. In one embodiment, a PD-1 inhibitor reduces the negative co-stimulatory signal mediated by or through cell surface proteins expressed on T lymphocytes mediated signaling through PD-1 so as render a dysfunctional T-cell less non-dysfunctional. In some embodiments, the PD-1 inhibitor is an anti-PD-1 antibody. In certain embodiments, the PD-1 inhibitor is selected from pembrolizumab, cemiplimab, tislelizumab, nivolumab, and combinations thereof. In one embodiment, the PD-1 inhibitor is pembrolizumab, or a biosimilar thereof. In one embodiment, the PD-1 inhibitor is cemiplimab, or a biosimilar thereof. In one embodiment, the PD-1 inhibitor is tislelizumab, or a biosimilar thereof. In one embodiment, the PD-1 inhibitor is nivolumab, or a biosimilar thereof.

The term “PD-L1 binding antagonist” as used herein refers to a PD-L1 inhibitor, i.e., a molecule that decreases, blocks, inhibits, abrogates or interferes with signal transduction resulting from the interaction of PD-L1 with either one or more of its binding partners, such as PD-1 and/or B7-1. In some embodiments, a PD-L1 inhibitor is a molecule that inhibits the binding of PD-L1 to its binding partners. In a specific aspect, the PD-L1 inhibitor inhibits binding of PD-L1 to PD-1 and/or B7-1. In some embodiments, the PD-L1 inhibitors include anti-PD-L1 antibodies, antigen binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1 and/or B7-1. In one embodiment, a PD-L1 inhibitor reduces the negative co-stimulatory signal mediated by or through cell surface proteins expressed on T lymphocytes mediated signaling through PD-L1 so as render a dysfunctional T-cell less non-dysfunctional. In some embodiments, a PD-L1 inhibitor is an anti-PD-L1 antibody. In certain embodiments, the PD-L1 inhibitor is selected from avelumab, atezolizumab, durvalumab, BMS-936559, and combinations thereof. In a specific aspect, an anti-PD-L1 inhibitor is avelumab or a biosimilar thereof. In another specific aspect, an anti-PD-L1 inhibitor is atezolizumab or a biosimilar thereof. In another specific aspect, an anti-PD-L1 inhibitor is durvalumab or a biosimilar thereof. In another specific aspect, an anti-PD-L1 inhibitor is BMS-936559 (MDX-1105) or a biosimilar thereof.

A “biosimilar” means an antibody or antigen-binding fragment that has the same primary amino acid sequence as compared to a reference antibody (e.g., nivolumab or pembrolizumab) and optionally, may have detectable differences in post-translation modifications (e.g., glycosylation and/or phosphorylation) as compared to the reference antibody (e.g., a different glycoform).

### PRMT5 Inhibitors

As provided above, the PRMT5 inhibitor is also administered in the methods of the disclosure. A “PRMT5 inhibitor” as used herein refers to compounds of the disclosure as described herein. These compounds are capable of negatively modulating or inhibiting all or a portion of the enzymatic activity of the PRMT5, particularly, in the presence of bound MTA in vitro or in vivo or in cells expressing elevated levels of MTA. In certain embodiments, the PRMT5 inhibitor is a MTA-cooperative PRMT5 inhibitor.

In certain embodiments, the PRMT5 inhibitor of the disclosure is any one of the PRMT5 inhibitors disclosed in International patent publication No. WO 2021/050915 A1, published 18 Mar. 2021, incorporated by reference in its entirety.

In certain other embodiments, the PRMT5 inhibitor of the disclosure is any one of the PRMT5 inhibitors disclosed International patent publication No. WO 2022/192745, published 15 Sep. 2022, incorporated by reference in its entirety.

In certain other embodiments, the PRMT5 inhibitor of the disclosure is any one of the PRMT5 inhibitors disclosed in International patent publication No. WO2023/081367, published 3 Aug. 2023, incorporated by reference in its entirety.

In certain other embodiments, the PRMT 5 inhibitor of the disclosure is any one of the PRMT5 inhibitors disclosed in International patent publication No. WO2023/278564, published 5 Jan. 2023, incorporated by reference in its entirety.

For example, the PRMT5 inhibitor in the methods of the disclosure as described herein is a compound of Formula IIA, IIB or IIC 1:

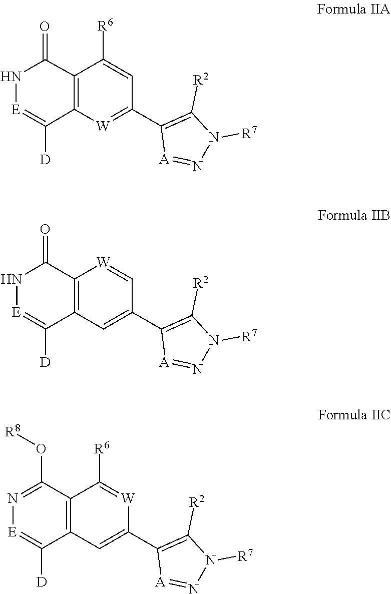

or a pharmaceutically acceptable salt thereof, wherein:
A is CR⁹ or N;
D is (C(R⁹)₂)₁₋₂—NH₂,

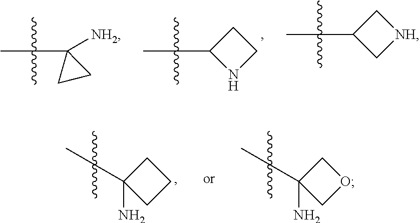 [SMILES: *C1(N)CC1 ; *C1CCN1 ; *C1CNC1 ; *C1(N)CCC1 ; *C1(N)COC1]

### or D is

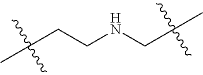 [SMILES: *CCNC*]

where the methylene is bonded to E where E is C;
E is C, CR⁹ or N;
each L is independently a bond or C₁-C₃ alkylene;
W is CR⁹ or N;
each X is independently a bond, O, S, —NR⁴— or —NR⁴C(O)—;
each Z is independently a bond, —SO—, —SO₂—, —CH(OH)— or —C(O)—;
each R² is independently hydroxy, halogen, cyano, cyanomethyl, —(NR⁴)₂, hydroxyalkyl, alkoxy, —SO₂C₁-C₃alkyl, —X-arC₁-C₃alkyl, heteroalkyl, C₂-C₄ alkynyl, —X-haloalkyl, —X—C₁-C₅ alkyl, —Z—C₁-C₅alkyl, heterocyclyl, —X-L-cycloalkyl, —Z-cycloalkyl, —X-aryl, —Z-aryl, or —X-heteroaryl, wherein the heterocyclyl, the cycloalkyl, the aryl and the heteroaryl are optionally substituted with one or more R⁵;
each R⁴ is independently hydrogen or C₁-C₃ alkyl;
each R⁵ is independently cyano, oxo, halogen, C₁-C₃ alkyl, hydroxyalkyl, hydroxy, alkoxy, alkoxy-C₁-C₃ alkyl, —X-haloalkyl, —Z-cycloalkyl, —X-arC₁-C₃alkyl, —X-arC₁-C₃alkyl substituted with cyano, —X-L-cycloalkyl optionally substituted with C₁-C₃ alkyl or oxo, —X-L-heteroaryl optionally substituted with one or more C₁-C₃ alkyl or oxo, —X-L-heterocyclyl optionally substituted with one or more C₁-C₃ alkyl or oxo, or —X-aryl;
R⁶ is hydrogen, halogen, C₁-C₃ alkyl, haloalkyl, hydroxy, alkoxy, C₁-C₃ alkyl-alkoxy, N(R⁹)₂, NR⁹C(O)R⁹, C(O)R⁹, oxetane and THF;
R⁷ is H or C₁-C₃ alkyl optionally substituted with one or more halogen;
R⁸ is H or C₁-C₃ alkyl; and
each R⁹ is independently H or C₁-C₃ alkyl, halogen or haloalkyl.

Embodiment 2 provides the PRMT5 inhibitor in the methods of the disclosure as a compound of Formula IIA:

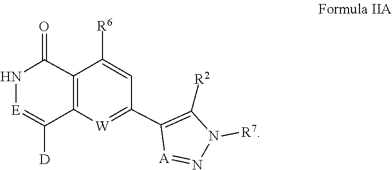

In certain embodiments, the PRMT5 inhibitor in the methods of the disclosure as a compound of Formula IIB:

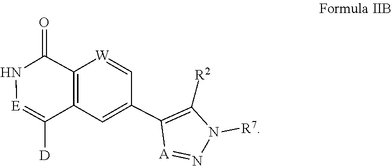

In certain embodiments, the PRMT5 inhibitor in the methods of the disclosure as a compound of Formula IIC:

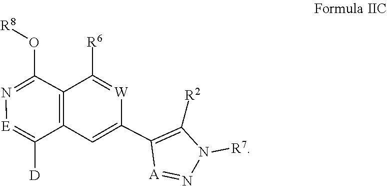

In certain embodiments of Formula IIA, IIB, and IIC, W is CR⁹.

In certain embodiments of Formula IIA, IIB, and IIC, A is CR⁹.

In certain embodiments of Formula IIA, IIB, and IIC, E is N.

In certain embodiments of Formula IIA, IIB, and IIC, W is CR⁹, A is CR⁹ and E is N.

Embodiment 9 provides the method of any of embodiments 1-8, wherein R² is selected from: benzothiophene, naphthalene, quinoline, chromane, isochromane, dihydrobenzodioxine, indolazine, tetrahydroindolazine, dihydroisobenzofuran, benzene, isoquinolinone, benzodioxone, thienopyridine, tetrahydroindolone, indolizine, dihydroindolizinone, imadazopyridinone, thienopyrimidine, thiophene, pyrrolopyrimidinone, thiazolopyridinone, dihydropyrrolizine, isoindalone and tetrahydroisoquinoline.

In certain embodiments of Formula IIA, IIB, and IIC, each R⁵ is independently cyano, oxo, halogen, C1-C3 alkyl, hydroxy, hydroxyalkyl, alkoxy-C1-C3alkyl, —X-L-heterocyclyl optionally substituted with one or more C1-C3alkyl or oxo, —X-L-cycloalkyl optionally substituted with C1-C3 alkyl or oxo.

In certain embodiments of Formula IIA, IIB, and IIC, R⁶ is selected from hydrogen, hydroxy, chlorine, —NHC(O)CH₃, —C(O)CF₂H, —NH₂, —CF₂, —CH₃, —O—CH₂CH₃, —CH₂—CH₂—O—CH₃, oxetane and THF.

In certain embodiments of Formula IIA, IIB, and IIC, one of L, X and Z is a bond. In certain embodiments, all of L, X and Z are bonds.

One aspect of the disclosure provides the method wherein the PRMT5 inhibitor is a compound of the Formula IIIC:

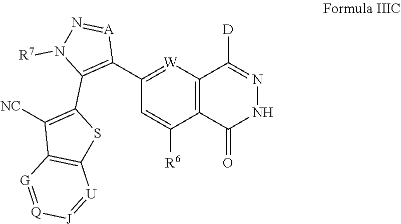

or a pharmaceutically acceptable salt thereof, wherein
A is CR⁹ or N;
D is —CH₂—NH₂,

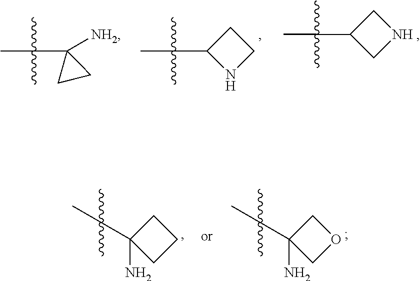 [SMILES: *C1(N)CC1 ; *C1CCN1 ; *C1CNC1 ; *C1(N)CCC1 ; *C1(N)COC1]

W is CR⁹ or N, where R⁹ is H or C₁-C₃ alkyl;
G, Q, J and U are independently selected from C(H), C(R⁵), and N, provided only one or two of G, Q, J, and U can be N;
   each R⁵ is independently hydroxy, halogen, C₁-C₆ alkyl, C₁-C₆ haloalkyl, C₁-C₆ alkoxy, C₁-C₆ haloalkoxy, C₃-C₆ cycloalkoxy, C₃-C₆ cycloalkyl, C₃-C₆ heterocycloalkyl, or C₁-C₃ alkoxyC₁-C₃ alkyl;
R⁶ is hydrogen, halogen, C₁-C₆ alkyl, C₁-C₆ haloalkyl, hydroxy, C₁-C₆ alkoxy, C₁-C₃ alkoxyC₁-C₃ alkyl, C₃-C₆ heterocycloalkyl, —C(O)—C₁-C₃ haloalkyl, —N(R⁹)₂, or —NR¹⁵(CO)R¹⁶,
   where each R⁹ is independently H or C₁-C₃ alkyl, R¹⁵ is hydrogen or methyl, and R¹⁶ is C₁-C₃ alkyl; and
R⁷ is C₁-C₃ alkyl or C₁-C₃ haloalkyl.

In certain embodiments of Formula IIIC, A is CH.

In certain embodiments of Formula IIIC, W is N.

In certain embodiments of Formula IIIC, W is CH.

In certain embodiments of Formula IIIC, D is —CH₂—NH₂.

In certain embodiments, the PRMT5 inhibitor is a compound according Formula IIIC having the formula:

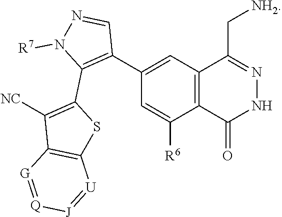

In certain embodiments of Formula IIIC, R⁶ is hydrogen, halogen, C₁-C₆ alkyl, C₁-C₆ haloalkyl, hydroxy, C₁-C₆ alkoxy, C₁-C₃ alkoxyC₁-C₃ alkyl, C₃-C₆ heterocycloalkyl, —C(O)—C₁-C₃ haloalkyl, —N(R⁹)₂, or —NR¹⁵(CO)R¹⁶.

In certain embodiments of Formula IIIC, R⁶ is hydrogen, halogen, C₁-C₃ alkyl, C₁-C₃ haloalkyl, hydroxy, C₁-C₃ alkoxy, C₁-C₃ alkoxyC₁-C₃ alkyl, C₃-C₆ heterocycloalkyl, —C(O)—C₁-C₃ haloalkyl, —N(R⁹)₂, or —NR¹⁵(CO)R¹⁶.

In certain embodiments of Formula IIIC, R⁶ is hydrogen, chloro, fluoro, methyl, ethyl, difluoromethyl, hydroxy, methoxy, ethoxy, (methoxy)methyl, (ethoxy)methyl, (methoxy)ethyl, (ethoxy)ethyl, oxetanyl, tetrahydrofuranyl, —C(O)-difluoromethyl, —NH₂, or —NH(CO)CH₃.

In certain embodiments of Formula IIIC, R⁶ is halogen, C₁-C₆ alkyl, C₁-C₆ haloalkyl, hydroxy, C₁-C₆ alkoxy, C₁-C₃ alkoxyC₁-C₃ alkyl, C₃-C₆ heterocycloalkyl, —C(O)—C₁-C₃ haloalkyl, —N(R⁹)₂, or —NR¹⁵(CO)R¹⁶.

In certain embodiments of Formula IIIC, R⁶ is halogen, C₁-C₃ alkyl, C₁-C₃ haloalkyl, hydroxy, C₁-C₃ alkoxy, C₁-C₃ alkoxyC₁-C₃ alkyl, C₃-C₆ heterocycloalkyl, —C(O)—C₁-C₃ haloalkyl, —N(R⁹)₂, or —NR¹⁵(CO)R¹⁶.

In certain embodiments of Formula IIIC, R⁶ is chloro, fluoro, methyl, ethyl, difluoromethyl, hydroxy, methoxy, ethoxy, (methoxy)methyl, (ethoxy)methyl, (methoxy)ethyl, (ethoxy)ethyl, oxetanyl, tetrahydrofuranyl, —C(O)-difluoromethyl, —NH₂, or —NH(CO)CH₃.

In certain embodiments of Formula IIIC, each G, Q, J and U is independently C(H).

In certain embodiments of Formula IIIC, G, Q, J and U are independently selected from C(H) and C(R⁵).

In certain embodiments of Formula IIIC, G, Q, J and U are independently selected from C(H) and N.

In certain embodiments of Formula IIIC,
R⁶ is hydrogen;
at least one of G, Q, J, and U is C(R⁵), and the remaining G, Q, J, and U are independently selected from C(H), C(R⁵) and N, wherein each R⁵ is independently hydroxy, halogen, C₁-C₆ alkyl, C₁-C₆ haloalkyl, C₁-C₆ alkoxy, C₁-C₆ haloalkoxy, C₃-C₆ cycloalkoxy, C₃-C₆ cycloalkyl, C₃-C₆ heterocycloalkyl, or C₁-C₃ alkoxyC₁-C₃ alkyl.

For example, in certain embodiments, one or two of G, Q, J and U is N.

In certain embodiments of Formula IIIC,
R⁶ is hydrogen;
at least one of G, Q, J, and U is C(R⁵), and the remaining G, Q, J, and U are independently selected from C(H) and C(R⁵), wherein each R⁵ is independently hydroxy, halogen, C₁-C₆ alkyl, C₁-C₆ haloalkyl, C₁-C₆ alkoxy, C₁-C₆ haloalkoxy, C₃-C₆ cycloalkoxy, C₃-C₆ cycloalkyl, C₃-C₆ heterocycloalkyl, or C₁-C₃ alkoxyC₁-C₃ alkyl.

For example, in certain embodiments, at least one of G, Q, J, and U is C(R⁵), and the remaining G, Q, J, and U are independently C(H); for example only one of G, Q, J, and U is C(R⁵). In certain embodiments, two of G, Q, J, and U is C(R⁵), and the remaining G, Q, J, and U are independently C(H). In certain embodiments, three of G, Q, J, and U is C(R⁵), and the remaining G, Q, J, and U is C(H).

In certain embodiments of Formula IIIC, G, Q, J, and U together with the thiophene to which they are attached form:

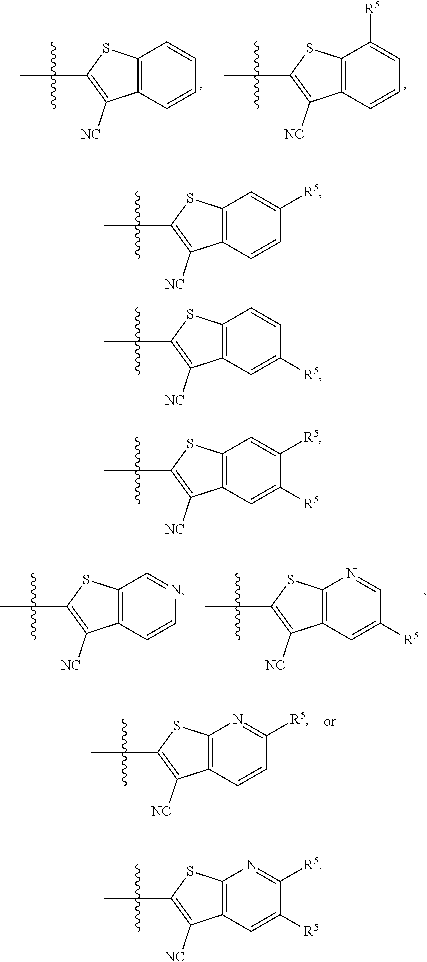

In certain embodiments of Formula IIIC, G, Q, J, and U together with the thiophene ring to which they are attached form a benzo[b]thiophene.

In certain embodiments of Formula IIIC, R⁵, if present, is hydroxy, halogen, C₁-C₃ alkyl, C₁-C₃ haloalkyl, C₁-C₃ alkoxy, C₁-C₃ haloalkoxy, C₃-C₆ cycloalkoxy, C₃-C₆ cycloalkyl, C₃-C₆ heterocycloalkyl, or C₁-C₃ alkoxyC₁-C₃ alkyl.

In certain embodiments of Formula IIIC, R⁵, if present, is hydroxy, halogen, C₁-C₃ alkyl, C₁-C₃ haloalkyl, C₁-C₃ alkoxy, C₁-C₃ haloalkoxy, C₃-C₆ heterocycloalkyl, or C₁-C₃ alkoxyC₁-C₃ alkyl.

In certain embodiments of Formula IIIC, R⁵, if present, is hydroxy, chloro, fluoro, methyl, ethyl, methoxy, ethoxy, 2,2-difluoroethoxy, oxetanyl, tetrahydrofuranyl, (methoxy)methyl, (ethoxy)methyl, (methoxy)ethyl, or (ethoxy)ethyl.

In certain embodiments of Formula IIIC, R⁷ is methyl.

In certain embodiments of Formula IIIC, R⁷ is ethyl.

In certain embodiments of Formula IIIC, R⁷ is propyl (e.g., isopropyl).

In certain embodiments of Formula IIIC, R⁷ is difluoromethyl or trifluoromethyl.

In certain embodiments of Formula IIIC, the PRMT5 inhibitor is of the formula:

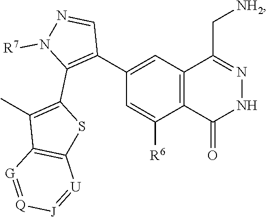

wherein
G, Q, J, and U together with the thiophene to which they are attached form:

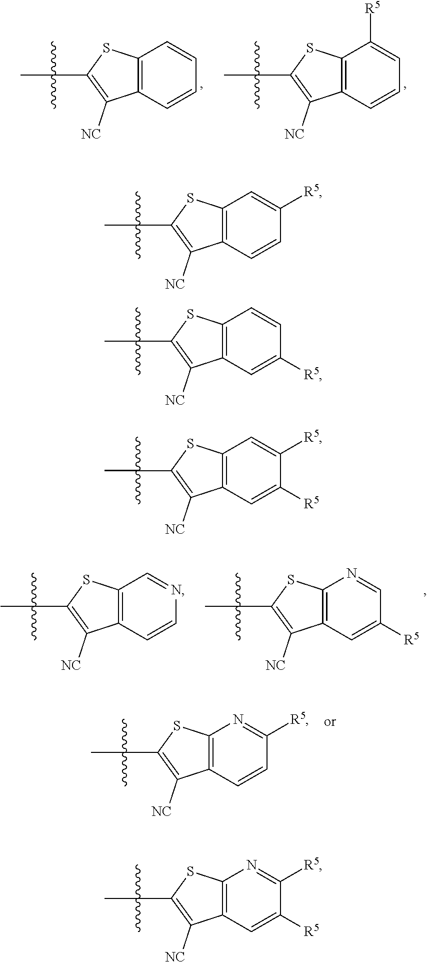

where each R⁵ is independently hydroxy, halogen, C₁-C₃ alkyl, C₁-C₃ haloalkyl, C₁-C₃ alkoxy, C₁-C₃ haloalkoxy, C₃-C₆ heterocycloalkyl, or C₁-C₃ alkoxyC₁-C₃ alkyl; and
R⁶ is hydrogen, halogen, C₁-C₃ alkyl, C₁-C₃ haloalkyl, hydroxy, C₁-C₃ alkoxy, C₁-C₃ alkoxyC₁-C₃ alkyl, C₃-C₆ heterocycloalkyl, —C(O)—C₁-C₃ haloalkyl, —N(R⁹)₂, or —NR¹⁵(CO)R¹⁶.

In certain embodiments of Formula IIIC, the PRMT5 inhibitor is of the formula:

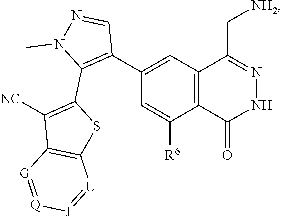

wherein
G, Q, J, and U together with the thiophene to which they are attached form:

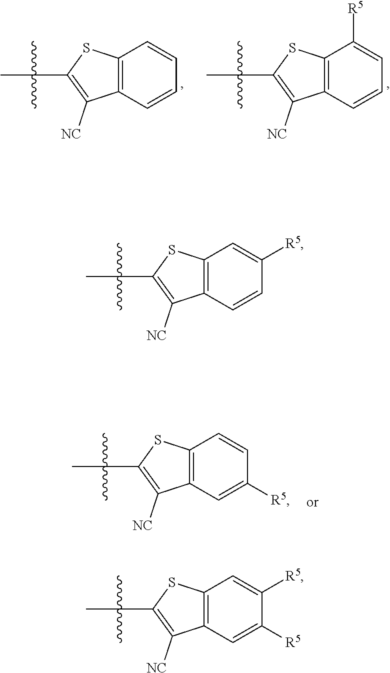

where each R⁵ is independently hydroxy, halogen, C₁-C₃ alkyl, C₁-C₃ haloalkyl, C₁-C₃ alkoxy, C₁-C₃ haloalkoxy, C₃-C₆ heterocycloalkyl, or C₁-C₃ alkoxyC₁-C₃ alkyl; and
R⁶ is halogen, C₁-C₃ alkyl, C₁-C₃ haloalkyl, hydroxy, C₁-C₃ alkoxy, C₁-C₃ alkoxyC₁-C₃ alkyl, C₃-C₆ heterocycloalkyl, —C(O)—C₁-C₃ haloalkyl, —N(R⁹)₂, or —NR¹⁵(CO)R¹⁶.

In certain embodiments of Formula IIIC, the PRMT5 inhibitor is of the formula:

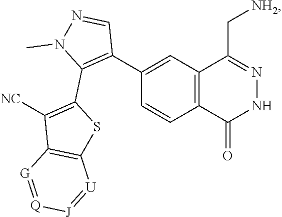

wherein
G, Q, J, and U together with the thiophene to which they are attached form:

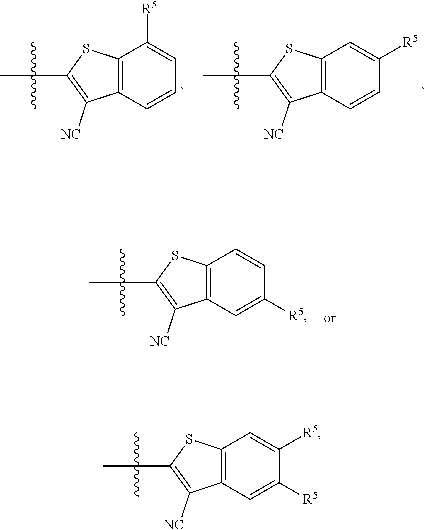

where each R⁵ is independently hydroxy, halogen, C₁-C₃ alkyl, C₁-C₃ haloalkyl, C₁-C₃ alkoxy, C₁-C₃ haloalkoxy, C₃-C₆ heterocycloalkyl, or C₁-C₃ alkoxyC₁-C₃ alkyl.

In certain embodiments of the methods as described herein, the PRMT5 inhibitor is: the PRMT5 inhibitor is:

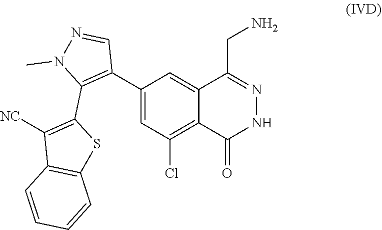 [SMILES: Cn1ncc(-c2cc(Cl)c3c(=O)[nH]nc(CN)c3c2)c1-c1sc2ccccc2c1C#N]

or a pharmaceutically acceptable salt thereof.

In certain embodiments, the PRMT5 inhibitor is a compound of the Formula IIIB:

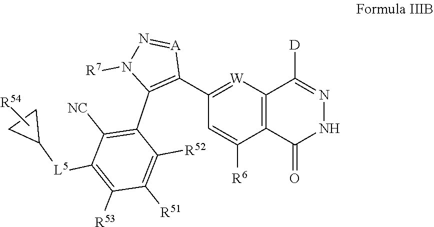

or a pharmaceutically acceptable salt thereof, wherein
A is CR⁹ or N;
D is —CH₂—NH₂,

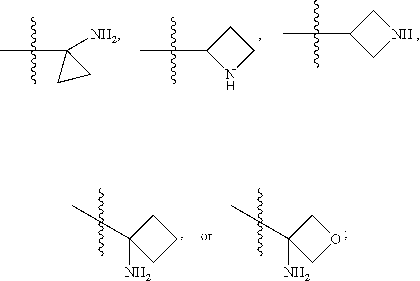 [SMILES: *C1(N)CC1 ; *C1CCN1 ; *C1CNC1 ; *C1(N)CCC1 ; *C1(N)COC1]

W is CR⁹ or N, where R⁹ is H or C₁-C₃ alkyl;
R⁵¹ is hydrogen, fluoro, chloro, or methyl, or R⁵¹ and R⁵² together with atoms to which they are attached form a C₄-C₆ heterocycloalkyl (e.g, hydrofuranyl);
R⁵² is fluoro, chloro, or methyl, or R⁵² and R⁵³ together with atoms to which they are attached form a phenyl;
R⁵³ is hydrogen, fluoro, chloro, or methyl;
R⁵⁴ is hydrogen, halogen, C₁-C₃ alkyl, or C₁-C₃ alkoxy;
L⁵ is —O— or —CH₂—;
R⁶ is hydrogen, halogen, C₁-C₆ alkyl, hydroxy, C₁-C₆ alkoxy, C₁-C₃ alkoxyC₁-C₃ alkyl, C₃-C₆ heterocycloalkyl, —C(O)—C₁-C₃ haloalkyl, or —NR¹⁵(CO)R¹⁶, where R¹⁵ is hydrogen or methyl, and R¹⁶ is C₁-C₃ alkyl;
R⁷ is C₁-C₃ alkyl or C₁-C₃ haloalkyl.

In certain embodiments of Formula IIIB:
A is —CH or —CCH₃;
D is —CH₂—NH₂;
W is —CH, —CCH₃, or N;
R⁵¹, R⁵², R⁵³, and R⁵⁴ are each independently selected from hydrogen, fluoro, chloro, or methyl;
L⁵ is —O—;
R⁶ is hydrogen, fluoro, chloro, or methyl; and
R⁷ is C₁-C₂ alkyl or C₁-C₂ haloalkyl.

In certain embodiments of Formula IIIB:
A and W are —CH;
D is —CH₂—NH₂;
R⁵¹, R⁵², and R⁵³ are each independently selected from hydrogen, fluoro, chloro, and methyl;
R⁵⁴ is hydrogen;
L⁵ is —O—;
R⁶ is hydrogen; and
R⁷ is methyl.

In certain embodiments of Formula IIIB:
A and W are —CH;
D is —CH₂—NH₂;
R⁵¹ and R⁵² are each independently selected from fluoro, chloro, and methyl;
R⁵³ and R⁵⁴ are hydrogen;
L⁵ is —O—;
R⁶ is hydrogen; and
R⁷ is methyl.

In certain embodiments of Formula IIIB, A is CH.

In certain embodiments of Formula IIIB, W is N.

In certain embodiments of Formula IIIB, W is CH.

In certain embodiments of Formula IIIB, D is —CH₂—NH₂.

In certain embodiments of Formula IIIB, R⁵⁴ is hydrogen or methyl.

In certain embodiments of Formula IIIB, R⁵⁴ is hydrogen.

In certain embodiments of Formula IIIB, R⁵⁴ is methyl.

In certain embodiments of Formula IIIB, the PRMT5 inhibitor is of the formula:

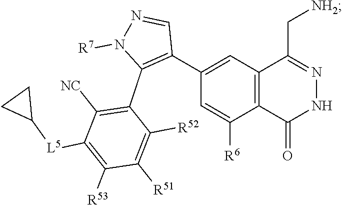

such as e.g.,

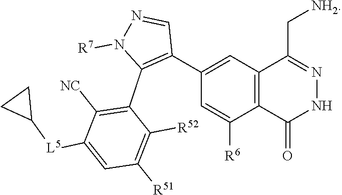

In certain embodiments of Formula IIIB, L⁵ is —CH₂—.

In certain embodiments of Formula IIIB, L⁵ is —O—.

In certain embodiments of Formula IIIB, R⁶ is hydrogen, halogen, C₁-C₃ alkyl, C₁-C₃ haloalkyl, hydroxy, C₁-C₃ alkoxy, C₁-C₃ alkoxyC₁-C₃ alkyl, C₃-C₆ heterocycloalkyl, —C(O)—C₁-C₃ haloalkyl, —N(R⁹)₂, or —NR¹⁵(CO)R¹⁶; for example, wherein R⁶ is hydrogen, chloro, fluoro, methyl, ethyl, difluoromethyl, hydroxy, methoxy, ethoxy, (methoxy)methyl, (ethoxy)methyl, (methoxy)ethyl, (ethoxy)ethyl, oxetanyl, tetrahydrofuranyl, —C(O)— difluoromethyl, —NH₂, or —NH(CO)CH₃.

In certain embodiments of Formula IIIB, R⁶ is hydrogen, halogen, C₁-C₆ alkyl, or C₁-C₆ alkoxy; for example, R⁶ is hydrogen, halogen, C₁-C₃ alkyl, or C₁-C₃ alkoxy.

In certain embodiments of Formula IIIB, R⁶ is hydrogen, chloro, fluoro, methyl, ethyl, methoxy, or ethoxy.

In certain embodiments of Formula IIIB, R⁶ is halogen, C₁-C₃ alkyl, C₁-C₃ haloalkyl, hydroxy, C₁-C₃ alkoxy, C₁-C₃ alkoxyC₁-C₃ alkyl, C₃-C₆ heterocycloalkyl, —C(O)—C₁-C₃ haloalkyl, —N(R⁹)₂, or —NR¹⁵(CO)R¹⁶; for example, wherein R⁶ is chloro, fluoro, methyl, ethyl, difluoromethyl, hydroxy, methoxy, ethoxy, (methoxy)methyl, (ethoxy)methyl, (methoxy)ethyl, (ethoxy)ethyl, oxetanyl, tetrahydrofuranyl, —C(O)-difluoromethyl, —NH₂, or —NH(CO)CH₃.

In certain embodiments of Formula IIIB, R⁶ is halogen, C₁-C₆ alkyl, or C₁-C₆ alkoxy; for example, R⁶ is halogen, C₁-C₃ alkyl, or C₁-C₃ alkoxy.

In certain embodiments of Formula IIIB, R⁶ is chloro, fluoro, methyl, ethyl, methoxy, or ethoxy.

In certain embodiments of Formula IIIB, R⁷ is methyl.

In certain embodiments of Formula IIIB, R⁷ is ethyl.

In certain embodiments of Formula IIIB, R⁷ is propyl (e.g., isopropyl).

In certain embodiments of Formula IIIB, R⁷ is difluoromethyl or trifluoromethyl.

In certain embodiments of Formula IIIB, R⁵³ is hydrogen or methoxy; or wherein R⁵³ is hydrogen.

In certain embodiments of Formula IIIB, the PRMT5 inhibitor is of the formula:

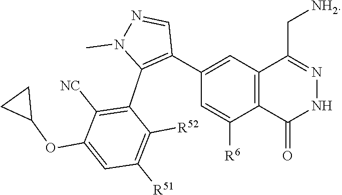

In certain embodiments, R⁵² is fluoro, and R⁵¹ is hydrogen, fluoro, chloro, or methyl.

In certain embodiments of Formula IIIB, R⁵² is fluoro, and R⁵¹ is chloro.

In certain embodiments of Formula IIIB, R⁵² is fluoro, and R⁵¹ is methyl or hydrogen (for example, R⁵² is fluoro and R⁵¹ is methyl; or R⁵² is fluoro and R⁵¹ is hydrogen).

In certain embodiments of Formula IIIB, R⁵¹ and R⁵² together with atoms to which they are attached form a hydrofuranyl

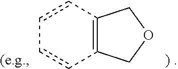

In certain embodiments of Formula IIIB, the PRMT5 inhibitor is

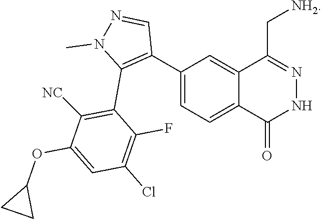 [SMILES: Cn1ncc(-c2ccc3c(=O)[nH]nc(CN)c3c2)c1-c1c(F)c(Cl)cc(OC2CC2)c1C#N]

In certain embodiments of Formula IIIB, the PRMT5 inhibitor is

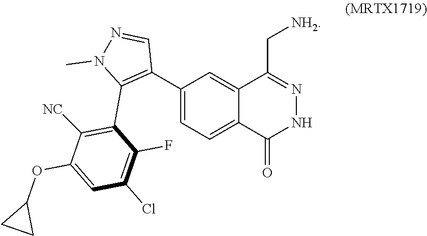

One aspect of the disclosure provides the method wherein the PRMT5 inhibitor is a compound of the Formula IIIA:

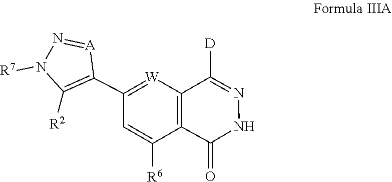

or a pharmaceutically acceptable salt thereof, wherein
A is CR⁹ or N;
D is —CH₂—NH₂,

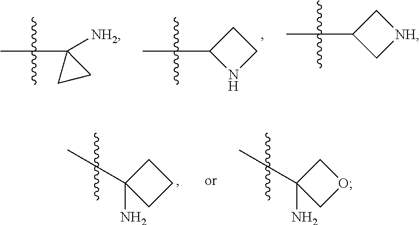 [SMILES: *C1(N)CC1 ; *C1CCN1 ; *C1CNC1 ; *C1(N)CCC1 ; *C1(N)COC1]

W is CR⁹ or N, where R⁹ is H or C₁-C₃ alkyl;
R² is

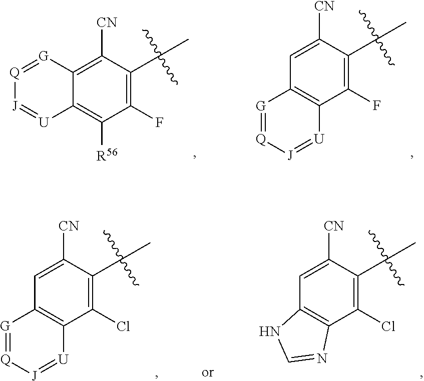

where R⁵⁶ is hydrogen, fluoro, chloro, or methyl,
G, Q, J and U are independently selected from C(H), C(R⁵), and N, provided only one or two of G, Q, J, and U can be N;
   each R⁵ is independently hydroxy, halogen, C₁-C₆ alkyl, C₁-C₆ haloalkyl, C₁-C₆ alkoxy, C₁-C₆ haloalkoxy, C₃-C₆ cycloalkoxy, C₃-C₆ cycloalkyl, C₃-C₆ heterocycloalkyl, or C₁-C₃ alkoxyC₁-C₃ alkyl;
R⁶ is hydrogen, halogen, C₁-C₆ alkyl, hydroxy, C₁-C₆ alkoxy, C₁-C₃ alkoxyC₁-C₃ alkyl, C₃-C₆ heterocycloalkyl, —C(O)—C₁-C₃ haloalkyl, or —NR¹⁵(CO)R¹⁶, where R¹⁵ is hydrogen or methyl, and R¹⁶ is C₁-C₃ alkyl; and
R⁷ is C₁-C₃ alkyl or C₁-C₃ haloalkyl.

One aspect of the disclosure provides the method wherein the PRMT5 inhibitor is a compound of the Formula IIIA:

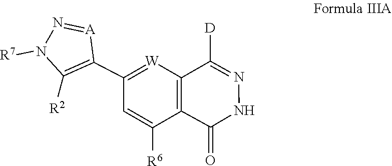

or a pharmaceutically acceptable salt thereof, wherein
A is CR⁹ or N;
D is —CH₂—NH₂,

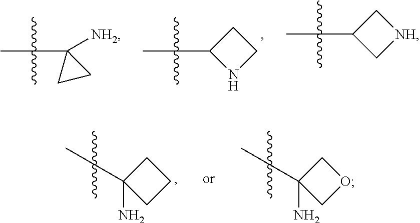 [SMILES: *C1(N)CC1 ; *C1CCN1 ; *C1CNC1 ; *C1(N)CCC1 ; *C1(N)COC1]

W is CR⁹ or N, where R⁹ is H or C₁-C₃ alkyl;
R² is

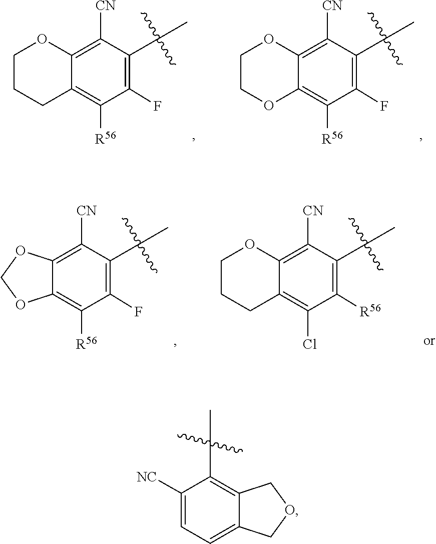

where R⁵⁶ is hydrogen, halogen, C₁-C₆ alkyl, C₁-C₆ haloalkyl, C₁-C₆ alkoxy, or C₁-C₆ haloalkoxy;
R⁶ is hydrogen, halogen, C₁-C₆ alkyl, hydroxy, C₁-C₆ alkoxy, C₁-C₃ alkoxyC₁-C₃ alkyl, C₃-C₆ heterocycloalkyl, —C(O)—C₁-C₃ haloalkyl, or —NR¹⁵(CO)R¹⁶, where R¹⁵ is hydrogen or methyl, and R¹⁶ is C₁-C₃ alkyl; and
R⁷ is C₁-C₃ alkyl or C₁-C₃ haloalkyl.

In certain embodiments of Formula IIIA, A is CH.

In certain embodiments of Formula IIIA, W is N.

In certain embodiments of Formula IIIA, W is CH.

In certain embodiments of Formula IIIA, D is —CH₂—NH₂.

In certain embodiments of Formula IIIA, the PRMT5 inhibitor is of the formula:

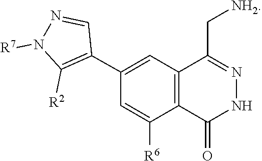

In certain embodiments of Formula IIIA, R² is

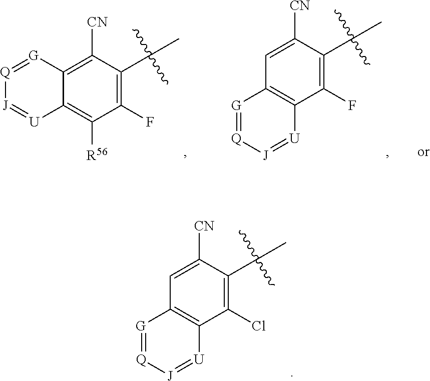

In certain embodiments of Formula IIIA, G, Q, J and U are independently selected from C(H) and C(R⁵).

In certain embodiments of Formula IIIA, G, Q, J and U are independently C(H).

In certain embodiments of Formula IIIA, at least one of G, Q, J, and U is C(R⁵), and the remaining G, Q, J, and U are independently C(H); for example only one of G, Q, J, and U is C(R⁵).

In certain embodiments of Formula IIIA, U is N, and G, Q, and J are independently selected from C(H) and C(R⁵).

In certain embodiments of Formula IIIA, G is N, and Q, J, and U are independently selected from C(H) and C(R⁵).

In certain embodiments of Formula IIIA, R⁵, if present, is hydroxy, halogen, C₁-C₃ alkyl, C₁-C₃ haloalkyl, C₁-C₃ alkoxy, C₁-C₃ haloalkoxy, C₃-C₆ cycloalkoxy, C₃-C₆ cycloalkyl, C₃-C₆ heterocycloalkyl, or C₁-C₃ alkoxyC₁-C₃ alkyl.

In certain embodiments of Formula IIIA, R⁵, if present, is hydroxy, halogen, C₁-C₃ alkyl, C₁-C₃ haloalkyl, C₁-C₃ alkoxy, C₁-C₃ haloalkoxy, C₃-C₆ heterocycloalkyl, or C₁-C₃ alkoxyC₁-C₃ alkyl.

In certain embodiments of Formula IIIA, R⁵, if present, is hydroxy, chloro, fluoro, methyl, ethyl, methoxy, ethoxy, 2,2-difluoroethoxy, oxetanyl, tetrahydrofuranyl, (methoxy)methyl, (ethoxy)methyl, (methoxy)ethyl, or (ethoxy)ethyl.

In certain embodiments of Formula IIIA, R⁵, if present, is halogen, C₁-C₆ alkyl, or C₁-C₆ alkoxy; for example, R⁶ is halogen, C₁-C₃ alkyl, or C₁-C₃ alkoxy.

In certain embodiments of Formula IIIA, R⁵, if present, is chloro, fluoro, methyl, ethyl, methoxy, or ethoxy.

In certain embodiments of Formula IIIA, R⁵⁶ is fluoro, chloro, or methyl.

In certain embodiments of Formula IIIA, R² is

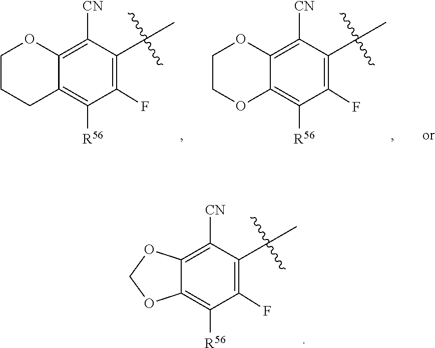

In certain embodiments of Formula IIIA, R⁵⁶ is hydrogen, fluoro, chloro, or methyl.

In certain embodiments of Formula IIIA, R⁶ is hydrogen, halogen, C₁-C₃ alkyl, C₁-C₃ haloalkyl, hydroxy, C₁-C₃ alkoxy, C₁-C₃ alkoxyC₁-C₃ alkyl, C₃-C₆ heterocycloalkyl, —C(O)—C₁-C₃ haloalkyl, —N(R⁹)₂, or —NR¹⁵(CO)R¹⁶; for example, wherein R⁶ is hydrogen, chloro, fluoro, methyl, ethyl, difluoromethyl, hydroxy, methoxy, ethoxy, (methoxy)methyl, (ethoxy)methyl, (methoxy)ethyl, (ethoxy)ethyl, oxetanyl, tetrahydrofuranyl, —C(O)-difluoromethyl, —NH₂, or —NH(CO)CH₃.

In certain embodiments of Formula IIIA, R⁶ is hydrogen, halogen, C₁-C₆ alkyl, or C₁-C₆ alkoxy; for example, R⁶ is hydrogen, halogen, C₁-C₃ alkyl, or C₁-C₃ alkoxy.

In certain embodiments of Formula IIIA, R⁶ is hydrogen, chloro, fluoro, methyl, ethyl, methoxy, or ethoxy.

In certain embodiments of Formula IIIA, R⁶ is halogen, C₁-C₃ alkyl, C₁-C₃ haloalkyl, hydroxy, C₁-C₃ alkoxy, C₁-C₃ alkoxyC₁-C₃ alkyl, C₃-C₆ heterocycloalkyl, —C(O)—C₁-C₃ haloalkyl, —N(R⁹)², or —NR¹⁵(CO)R¹⁶; for example, wherein R⁶ is chloro, fluoro, methyl, ethyl, difluoromethyl, hydroxy, methoxy, ethoxy, (methoxy)methyl, (ethoxy)methyl, (methoxy)ethyl, (ethoxy)ethyl, oxetanyl, tetrahydrofuranyl, —C(O)-difluoromethyl, —NH₂, or —NH(CO)CH₃.

In certain embodiments of Formula IIIA, R⁶ is halogen, C₁-C₆ alkyl, or C₁-C₆ alkoxy; for example, R⁶ is halogen, C₁-C₃ alkyl, or C₁-C₃ alkoxy.

In certain embodiments of Formula IIIA, R⁶ is chloro, fluoro, methyl, ethyl, methoxy, or ethoxy.

In certain embodiments of Formula IIIA, R⁷ is methyl.

In certain embodiments of Formula IIIA, R⁷ is ethyl.

In certain embodiments of Formula IIIA, R⁷ is propyl (e.g., isopropyl).

In certain embodiments of Formula IIIA, R⁷ is difluoromethyl or trifluoromethyl.

In certain embodiments of the methods of the disclosure as described herein, the PRMT5 inhibitor is:

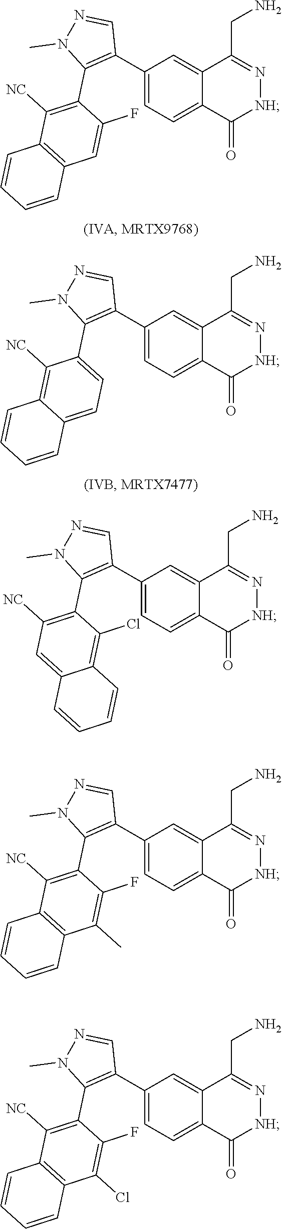 [SMILES: Cn1ncc(-c2ccc3c(=O)[nH]nc(CN)c3c2)c1-c1c(F)cc2ccccc2c1C#N ; Cn1ncc(-c2ccc3c(=O)[nH]nc(CN)c3c2)c1-c1ccc2ccccc2c1C#N ; Cn1ncc(-c2ccc3c(=O)[nH]nc(CN)c3c2)c1-c1c(C#N)cc2ccccc2c1Cl ; Cc1c(F)c(-c2c(-c3ccc4c(=O)[nH]nc(CN)c4c3)cnn2C)c(C#N)c2ccccc12 ; Cn1ncc(-c2ccc3c(=O)[nH]nc(CN)c3c2)c1-c1c(F)c(Cl)c2ccccc2c1C#N]

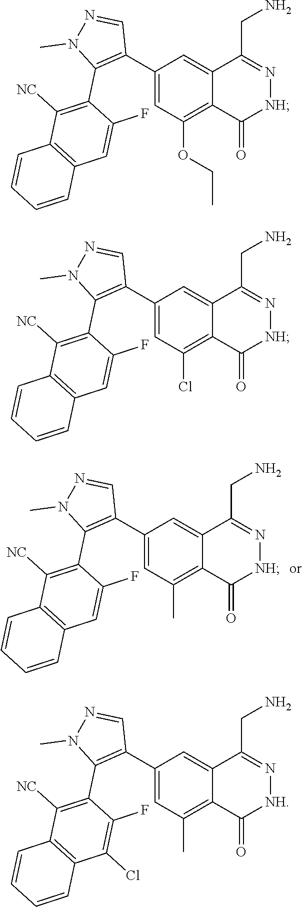 [SMILES: CCOc1cc(-c2cnn(C)c2-c2c(F)cc3ccccc3c2C#N)cc2c(CN)n[nH]c(=O)c12 ; Cn1ncc(-c2cc(Cl)c3c(=O)[nH]nc(CN)c3c2)c1-c1c(F)cc2ccccc2c1C#N ; Cc1cc(-c2cnn(C)c2-c2c(F)cc3ccccc3c2C#N)cc2c(CN)n[nH]c(=O)c12 ; Cc1cc(-c2cnn(C)c2-c2c(F)c(Cl)c3ccccc3c2C#N)cc2c(CN)n[nH]c(=O)c12]

In certain embodiments of the methods of the disclosure as described herein, the PRMT5 inhibitor is:

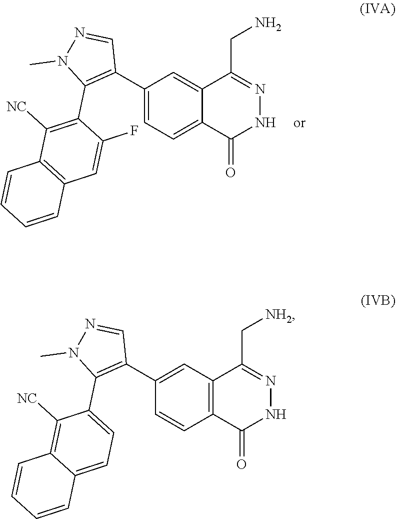 [SMILES: Cn1ncc(-c2ccc3c(=O)[nH]nc(CN)c3c2)c1-c1c(F)cc2ccccc2c1C#N ; Cn1ncc(-c2ccc3c(=O)[nH]nc(CN)c3c2)c1-c1ccc2ccccc2c1C#N]

or a pharmaceutically acceptable salt thereof.

In certain embodiments of the methods of the disclosure as described herein, the PRMT5 inhibitor is:

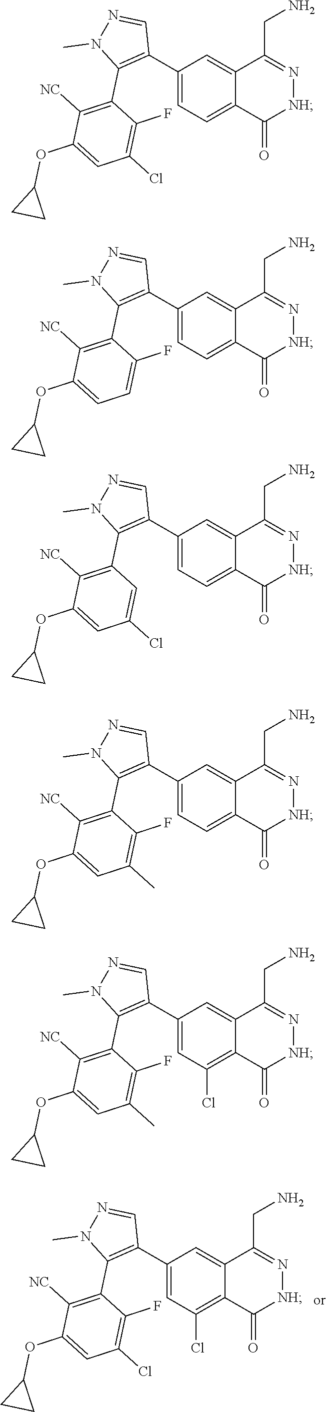 [SMILES: Cn1ncc(-c2ccc3c(=O)[nH]nc(CN)c3c2)c1-c1c(F)c(Cl)cc(OC2CC2)c1C#N ; Cn1ncc(-c2ccc3c(=O)[nH]nc(CN)c3c2)c1-c1c(F)ccc(OC2CC2)c1C#N ; Cn1ncc(-c2ccc3c(=O)[nH]nc(CN)c3c2)c1-c1cc(Cl)cc(OC2CC2)c1C#N ; Cc1cc(OC2CC2)c(C#N)c(-c2c(-c3ccc4c(=O)[nH]nc(CN)c4c3)cnn2C)c1F ; Cc1cc(OC2CC2)c(C#N)c(-c2c(-c3cc(Cl)c4c(=O)[nH]nc(CN)c4c3)cnn2C)c1F ; Cn1ncc(-c2cc(Cl)c3c(=O)[nH]nc(CN)c3c2)c1-c1c(F)c(Cl)cc(OC2CC2)c1C#N]

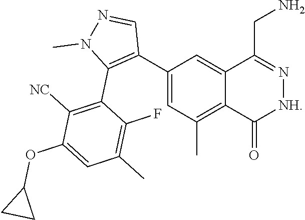 [SMILES: Cc1cc(OC2CC2)c(C#N)c(-c2c(-c3cc(C)c4c(=O)[nH]nc(CN)c4c3)cnn2C)c1F]

In certain embodiments of the methods of the disclosure as described herein, the PRMT5 inhibitor is:

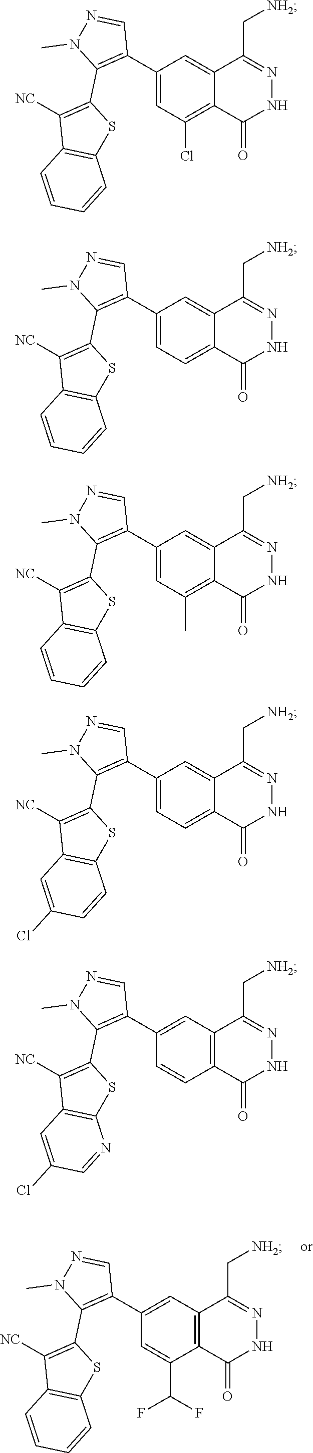

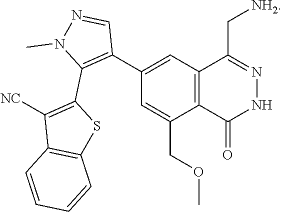 [SMILES: COCc1cc(-c2cnn(C)c2-c2sc3ccccc3c2C#N)cc2c(CN)n[nH]c(=O)c12]

In certain embodiments of the methods of the disclosure as described herein, the PRMT5 inhibitor is:

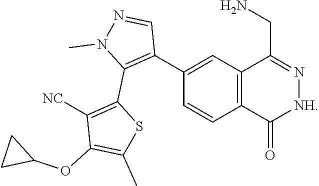 [SMILES: Cc1sc(-c2c(-c3ccc4c(=O)[nH]nc(CN)c4c3)cnn2C)c(C#N)c1OC1CC1]

In certain embodiments as described herein, the PRMT5 inhibitor is

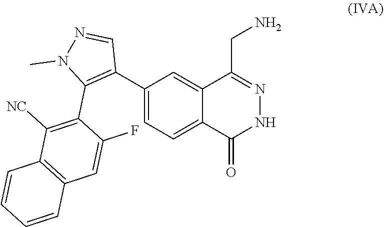 [SMILES: Cn1ncc(-c2ccc3c(=O)[nH]nc(CN)c3c2)c1-c1c(F)cc2ccccc2c1C#N]

or a pharmaceutically acceptable salt, and the immuno-oncology checkpoint inhibitor is a CTLA4 inhibitor. In other embodiments, the PRMT5 inhibitor is

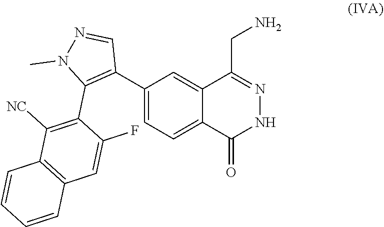 [SMILES: Cn1ncc(-c2ccc3c(=O)[nH]nc(CN)c3c2)c1-c1c(F)cc2ccccc2c1C#N]

or a pharmaceutically acceptable salt, and immuno-oncology checkpoint inhibitor is a PD-1 inhibitor. In certain other embodiments, the PRMT5 inhibitor is

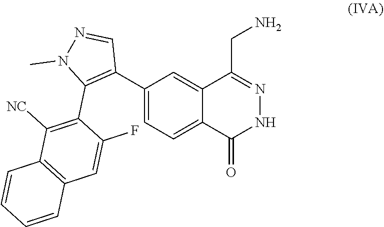 [SMILES: Cn1ncc(-c2ccc3c(=O)[nH]nc(CN)c3c2)c1-c1c(F)cc2ccccc2c1C#N]

or a pharmaceutically acceptable salt, and immuno-oncology checkpoint inhibitor is a PD-L1 inhibitor.

In certain embodiments as described herein, the PRMT5 inhibitor is

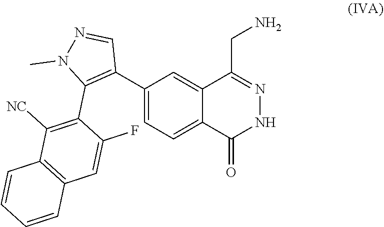 [SMILES: Cn1ncc(-c2ccc3c(=O)[nH]nc(CN)c3c2)c1-c1c(F)cc2ccccc2c1C#N]

or a pharmaceutically acceptable salt, and the immuno-oncology checkpoint inhibitor comprises a CTLA4 inhibitor and PD-1 inhibitor. In certain other embodiments, the PRMT5 inhibitor is

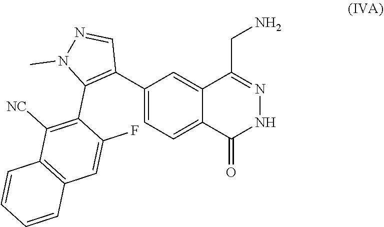 [SMILES: Cn1ncc(-c2ccc3c(=O)[nH]nc(CN)c3c2)c1-c1c(F)cc2ccccc2c1C#N]

or a pharmaceutically acceptable salt, and immuno-oncology checkpoint inhibitor comprises a CTLA4 inhibitor and PD-L1 inhibitor.

In certain embodiments of the methods as described herein, the PRMT5 inhibitor is

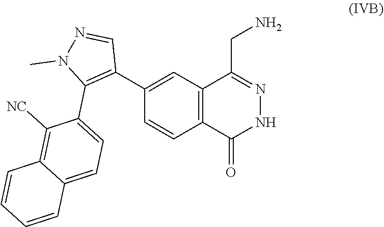 [SMILES: Cn1ncc(-c2ccc3c(=O)[nH]nc(CN)c3c2)c1-c1ccc2ccccc2c1C#N]

or a pharmaceutically acceptable salt, and the immuno-oncology checkpoint inhibitor is a CTLA4 inhibitor. In other embodiments, the PRMT5 inhibitor is

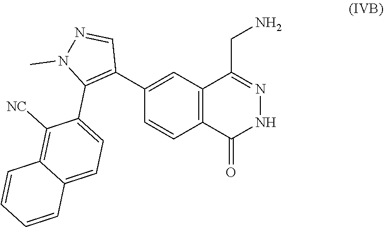 [SMILES: Cn1ncc(-c2ccc3c(=O)[nH]nc(CN)c3c2)c1-c1ccc2ccccc2c1C#N]

or a pharmaceutically acceptable salt, and the immuno-oncology checkpoint inhibitor is a PD-1 inhibitor. In certain other embodiments, the PRMT5 inhibitor is

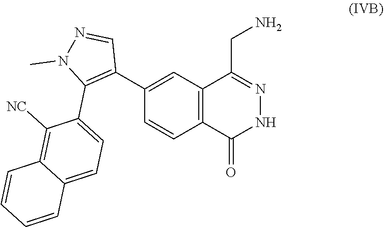 [SMILES: Cn1ncc(-c2ccc3c(=O)[nH]nc(CN)c3c2)c1-c1ccc2ccccc2c1C#N]

or a pharmaceutically acceptable salt, and the immuno-oncology checkpoint inhibitor is a PDL-1 inhibitor.

In certain embodiments of the method as described herein, the PRMT5 inhibitor is

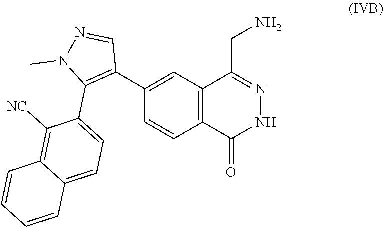 [SMILES: Cn1ncc(-c2ccc3c(=O)[nH]nc(CN)c3c2)c1-c1ccc2ccccc2c1C#N]

or a pharmaceutically acceptable salt, and the immuno-oncology checkpoint inhibitor comprises a CTLA4 inhibitor and a PD-1 inhibitor. In certain embodiments as described herein, the PRMT5 inhibitor is

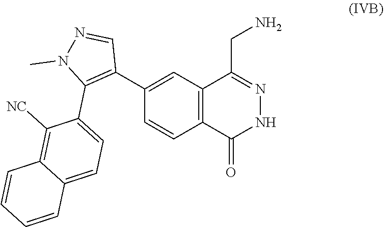 [SMILES: Cn1ncc(-c2ccc3c(=O)[nH]nc(CN)c3c2)c1-c1ccc2ccccc2c1C#N]

or a pharmaceutically acceptable salt, and the immuno-oncology checkpoint inhibitor comprises a CTLA4 inhibitor and a PD-L1 inhibitor.

In certain embodiments of the method as described herein, the PRMT5 inhibitor is

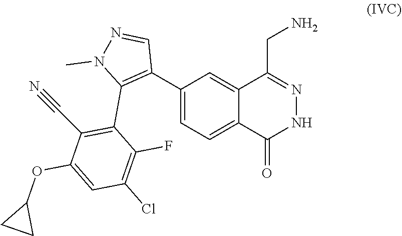 [SMILES: Cn1ncc(-c2ccc3c(=O)[nH]nc(CN)c3c2)c1-c1c(F)c(Cl)cc(OC2CC2)c1C#N]

or a pharmaceutically acceptable salt thereof, and the immuno-oncology checkpoint inhibitor is a CTLA4 inhibitor. In other embodiments, the PRMT5 inhibitor is

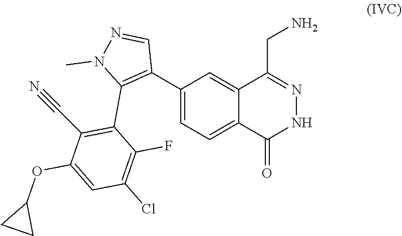 [SMILES: Cn1ncc(-c2ccc3c(=O)[nH]nc(CN)c3c2)c1-c1c(F)c(Cl)cc(OC2CC2)c1C#N]

or a pharmaceutically acceptable salt thereof, and the immuno-oncology checkpoint inhibitor is a PD-1 inhibitor. In certain other embodiments, the PRMT5 inhibitor is

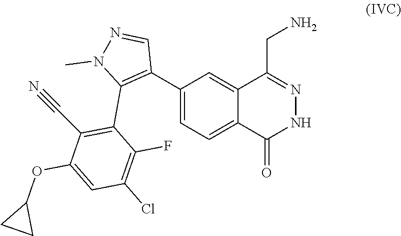 [SMILES: Cn1ncc(-c2ccc3c(=O)[nH]nc(CN)c3c2)c1-c1c(F)c(Cl)cc(OC2CC2)c1C#N]

or a pharmaceutically acceptable salt thereof, and the immuno-oncology checkpoint inhibitor is a PD-L1 inhibitor.

In certain embodiments of the methods as described herein, the PRMT5 inhibitor is

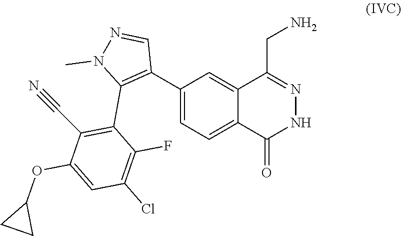 [SMILES: Cn1ncc(-c2ccc3c(=O)[nH]nc(CN)c3c2)c1-c1c(F)c(Cl)cc(OC2CC2)c1C#N]

or a pharmaceutically acceptable salt thereof, and the immuno-oncology checkpoint inhibitor comprises a CTLA4 inhibitor and a PD-1 inhibitor. In certain other embodiments of the methods as described herein, the PRMT5 inhibitor is

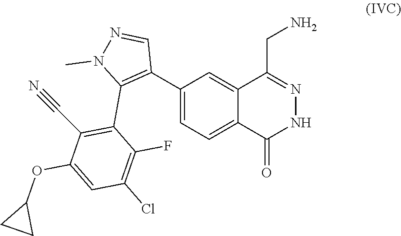 [SMILES: Cn1ncc(-c2ccc3c(=O)[nH]nc(CN)c3c2)c1-c1c(F)c(Cl)cc(OC2CC2)c1C#N]

or a pharmaceutically acceptable salt thereof, and the immuno-oncology checkpoint inhibitor comprises a CTLA4 inhibitor and a PD-L1 inhibitor.

In certain embodiments of the methods as described herein, the PRMT5 inhibitor is MRTX1719 or a pharmaceutically acceptable salt thereof, and the immuno-oncology checkpoint inhibitor is a CTLA4 inhibitor. In other embodiments, the PRMT5 inhibitor is MRTX1719 or a pharmaceutically acceptable salt thereof, and the immuno-oncology checkpoint inhibitor is a PD-1 inhibitor. In certain other embodiments, the PRMT5 inhibitor is MRTX1719 or a pharmaceutically acceptable salt thereof, and the immuno-oncology checkpoint inhibitor is a PD-L1 inhibitor.

In certain embodiments of the methods as described herein, the PRMT5 inhibitor is MRTX1719 or a pharmaceutically acceptable salt thereof, and the immuno-oncology checkpoint inhibitor comprises a CTLA4 inhibitor and a PD-1 inhibitor. In certain other embodiments, the PRMT5 inhibitor is MRTX1719 or a pharmaceutically acceptable salt thereof, and the immuno-oncology checkpoint inhibitor comprises a CTLA4 inhibitor and a PD-L1 inhibitor.

In certain embodiments of the method as described herein, the PRMT5 inhibitor is:

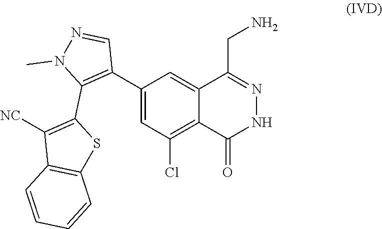 [SMILES: Cn1ncc(-c2cc(Cl)c3c(=O)[nH]nc(CN)c3c2)c1-c1sc2ccccc2c1C#N]

or a pharmaceutically acceptable salt thereof, and the immuno-oncology checkpoint inhibitor is a CTLA4 inhibitor. In other embodiments, the PRMT5 inhibitor is:

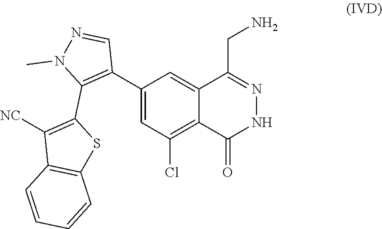 [SMILES: Cn1ncc(-c2cc(Cl)c3c(=O)[nH]nc(CN)c3c2)c1-c1sc2ccccc2c1C#N]

or a pharmaceutically acceptable salt thereof, the immuno-oncology checkpoint inhibitor is a PD-1 inhibitor. In certain other embodiments, the PRMT5 inhibitor is:

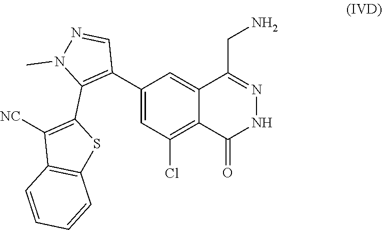 [SMILES: Cn1ncc(-c2cc(Cl)c3c(=O)[nH]nc(CN)c3c2)c1-c1sc2ccccc2c1C#N]

or a pharmaceutically acceptable salt thereof, the immuno-oncology checkpoint inhibitor is a PD-L1 inhibitor.

In certain embodiments of the method as described herein, the PRMT5 inhibitor is:

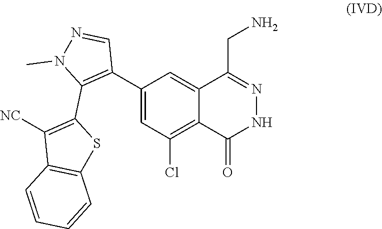 [SMILES: Cn1ncc(-c2cc(Cl)c3c(=O)[nH]nc(CN)c3c2)c1-c1sc2ccccc2c1C#N]

or a pharmaceutically acceptable salt thereof, the immuno-oncology checkpoint inhibitor comprises a CTLA4 inhibitor and a PD-1 inhibitor. In other embodiments as described herein, the PRMT5 inhibitor is:

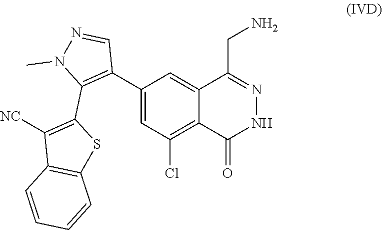 [SMILES: Cn1ncc(-c2cc(Cl)c3c(=O)[nH]nc(CN)c3c2)c1-c1sc2ccccc2c1C#N]

or a pharmaceutically acceptable salt thereof, the immuno-oncology checkpoint inhibitor comprises a CTLA4 inhibitor and a PD-L1 inhibitor.

### Definitions

For simplicity, chemical moieties are defined and referred to throughout primarily as univalent chemical moieties (e.g., alkyl, aryl, etc.). Nevertheless, such terms may also be used to convey corresponding multivalent moieties under the appropriate structural circumstances clear to those skilled in the art. For example, while an “alkyl” moiety generally refers to a monovalent radical (e.g. CH₃—CH₂—), in certain circumstances a bivalent linking moiety can be “alkyl,” in which case those skilled in the art will understand the alkyl to be a divalent radical (e.g., —CH₂—CH₂—), which is equivalent to the term “alkylene.” (Similarly, in circumstances in which a divalent moiety is required and is stated as being “aryl,” those skilled in the art will understand that the term “aryl” refers to the corresponding divalent moiety, arylene.) All atoms are understood to have their normal number of valences for bond formation (i.e., 4 for carbon, 3 for N, 2 for O, and 2, 4, or 6 for S, depending on the oxidation state of the S).

The term “amino” refers to —NH₂.

The term “acetyl” refers to “—C(O)CH₃.

As herein employed, the term “acyl” refers to an alkylcarbonyl or arylcarbonyl substituent wherein the alkyl and aryl portions are as defined herein.

The term “alkyl” as employed herein refers to saturated straight and branched chain aliphatic groups having from 1 to 12 carbon atoms. As such, “alkyl” encompasses C₁, C₂, C₃, C₄, C₅, C₆, C₇, C₈, C₉, C₁₀, C₁₁ and C₁₂ groups. Examples of alkyl groups include, without limitation, methyl, ethyl, propyl, isopropyl, butyl, isobutyl, sec-butyl, tert-butyl, pentyl, and hexyl.

The term “alkenyl” as used herein means an unsaturated straight or branched chain aliphatic group with one or more carbon-carbon double bonds, having from 2 to 12 carbon atoms. As such, “alkenyl” encompasses C₂, C₃, C₄, C₅, C₆, C₇, C₈, C₉, C₁₀, C₁₁ and C₁₂ groups.

Examples of alkenyl groups include, without limitation, ethenyl, propenyl, butenyl, pentenyl, and hexenyl.

The term “alkynyl” as used herein means an unsaturated straight or branched chain aliphatic group with one or more carbon-carbon triple bonds, having from 2 to 12 carbon atoms. As such, “alkynyl” encompasses C₂, C₃, C₄, C₅, C₆, C₇, C₈, C₉, C₁₀, C₁₁ and C₁₂ groups.

Examples of alkynyl groups include, without limitation, ethynyl, propynyl, butynyl, pentynyl, and hexynyl.

An “alkylene,” “alkenylene,” or “alkynylene” group is an alkyl, alkenyl, or alkynyl group, as defined hereinabove, that is positioned between and serves to connect two other chemical groups. Examples of alkylene groups include, without limitation, methylene, ethylene, propylene, and butylene. Exemplary alkenylene groups include, without limitation, ethenylene, propenylene, and butenylene. Exemplary alkynylene groups include, without limitation, ethynylene, propynylene, and butynylene.

The term “alkoxy” refers to —OC₁-C₆ alkyl.

The term “cycloalkyl” as employed herein is a saturated and partially unsaturated cyclic hydrocarbon group having 3 to 12 carbons. As such, “cycloalkyl” includes C₃, C₄, C₅, C₆, C₇, C₈, C₉, C₁₀, C₁₁ and C₁₂ cyclic hydrocarbon groups. Examples of cycloalkyl groups include, without limitation, cyclopropyl, cyclobutyl, cyclopentyl, cyclopentenyl, cyclohexyl, cyclohexenyl, cycloheptyl, and cyclooctyl.

The term “heteroalkyl” refers to an alkyl group, as defined hereinabove, wherein one or more carbon atoms in the chain are independently replaced O, S, or NR^{x}, wherein R^{x} is hydrogen or C₁-C₃ alkyl. Examples of heteroalkyl groups include methoxymethyl, methoxyethyl and methoxypropyl.

An “aryl” group is a C₆-C₁₄ aromatic moiety comprising one to three aromatic rings. As such, “aryl” includes C₆, C₁₀, C₁₃, and C₁₄ cyclic hydrocarbon groups. An exemplary aryl group is a C₆-C₁₀ aryl group. Particular aryl groups include, without limitation, phenyl, naphthyl, anthracenyl, and fluorenyl. An “aryl” group also includes fused multicyclic (e.g., bicyclic) ring systems in which one or more of the fused rings is non-aromatic, provided that at least one ring is aromatic, such as indenyl.

An “aralkyl” or “arylalkyl” group comprises an aryl group covalently linked to an alkyl group wherein the moiety is linked to another group via the alkyl moiety. An exemplary aralkyl group is —(C₁-C₆)alkyl(C₆-C₁₀) aryl, including, without limitation, benzyl, phenethyl, and naphthylmethyl. For example, an arC₁-C₃alkyl is an aryl group covalently linked to a C₁-C₃ alkyl.

A “heterocyclyl” or “heterocyclic” group is a mono- or bicyclic (fused or spiro) ring structure having from 3 to 12 atoms, (3, 4, 5, 6, 7, 8, 9, 10, 11 or 12 atoms), for example 4 to 8 atoms, wherein one or more ring atoms are independently —C(O)—, N, NR⁴, O, or S, and the remainder of the ring atoms are quaternary or carbonyl carbons. Examples of heterocyclic groups include, without limitation, epoxy, oxiranyl, oxetanyl, azetidinyl, aziridinyl, tetrahydrofuranyl, tetrahydropyranyl, tetrahydrothiophenyl, pyrrolidinyl, piperidinyl, piperazinyl, imidazolidinyl, thiazolidinyl, thiatanyl, dithianyl, trithianyl, azathianyl, oxathianyl, dioxolanyl, oxazolidinyl, oxazolidinonyl, decahydroquinolinyl, piperidonyl, 4-piperidonyl, thiomorpholinyl, dimethyl-morpholinyl, and morpholinyl. Specifically excluded from the scope of this term are compounds having adjacent ring O and/or S atoms.

As used herein, “L-heterocyclyl” refers to a heterocyclyl group covalently linked to another group via an alkylene linker.

As used herein, the term “heteroaryl” refers to a group having 5 to 14 ring atoms, preferably 5, 6, 10, 13 or 14 ring atoms; having 6, 10, or 14 π electrons shared in a cyclic array; and having, in addition to carbon atoms, from one to three heteroatoms that are each independently N, O, or S. Heteroaryl also includes fused multicyclic (e.g., bicyclic) ring systems in which one or more of the fused rings is non-aromatic, provided that at least one ring is aromatic and at least one ring contains an N, O, or S ring atom. Examples of heteroaryl groups include acridinyl, azocinyl, benzimidazolyl, benzofuranyl, benzo[d]oxazol-2(3H)-one, 2H-benzo[b][1,4]oxazin-3 (4H)-one, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, furanyl, furazanyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolinyl, 2H-pyrrolyl, pyrrolyl, quinazolinyl, quinolinyl, 4H-quinolizinyl, quinoxalinyl, quinuclidinyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl.

A “L-heteroaralkyl” or “L-heteroarylalkyl” group comprises a heteroaryl group covalently linked to another group via an alkylene linker. Examples of heteroalkyl groups comprise a C₁-C₆ alkyl group and a heteroaryl group having 5, 6, 9, or 10 ring atoms. Examples of heteroaralkyl groups include pyridylmethyl, pyridylethyl, pyrrolylmethyl, pyrrolylethyl, imidazolylmethyl, imidazolylethyl, thiazolylmethyl, thiazolylethyl, benzimidazolylmethyl, benzimidazolylethyl quinazolinylmethyl, quinolinylmethyl, quinolinylethyl, benzofuranylmethyl, indolinylethyl isoquinolinylmethyl, isoinodylmethyl, cinnolinylmethyl, and benzothiophenylethyl. Specifically excluded from the scope of this term are compounds having adjacent ring O and/or S atoms.

An “arylene,” “heteroarylene,” or “heterocyclylene” group is a bivalent aryl, heteroaryl, or heterocyclyl group, respectively, as defined hereinabove, that is positioned between and serves to connect two other chemical groups.

As employed herein, when a moiety (e.g., cycloalkyl, aryl, heteroaryl, heterocyclyl, urea, etc.) is described as “optionally substituted” without expressly stating the substituents it is meant that the group optionally has from one to four, preferably from one to three, more preferably one or two, non-hydrogen substituents.

The term “halogen” or “halo” as employed herein refers to chlorine, bromine, fluorine, or iodine.

The term “haloalkyl” refers to an alkyl chain in which one or more hydrogens have been replaced by a halogen. Exemplary haloalkyls are trifluoromethyl, difluoromethyl, flurochloromethyl, chloromethyl, and fluoromethyl.

The term “hydroxyalkyl” refers to -alkylene-OH.

### EXAMPLE

The methods of the disclosure are illustrated further by the following examples, which is not to be construed as limiting the disclosure in scope or spirit to the specific procedures and compounds described in them.

### MRTX1719+Immune Checkpoint Blockade Combination Study Procedure:

Immune-competent female BALB/c mice were implanted with murine syngeneic cancer model with a homozygous deletion of the Mtap gene (Mtap^{DEL}). Mouse health was monitored daily, and caliper measurements began when tumors were palpable. Tumor volume measurements were determined utilizing the formula 0.5×L×W2 in which L refers to length and W refers to width of each tumor. When tumors reached approximately 80 mm³, animals were randomized to receive A) vehicle (0.5% methylcellulose (4000 cps)/0.2% Tween80 in water) and isotype control antibodies), B) immune checkpoint blockade antibodies targeting mouse-Ctla-4 and mouse-Pd-1 and vehicle, C) a PRMT5 inhibitor and isotype control antibodies, or D) the PRMT5 inhibitor and immune checkpoint blockade antibodies targeting mouse-Ctla-4 and mouse-Pd-1. Tumor volumes were measured two or three times per week (n=10/treatment group). Tumor Growth Inhibition (% TGI) was calculated using the formula: (1−(Final Drug Treated Tumor Volume−Initial Drug Treated Tumor Volume)/(Final Vehicle Treated Tumor Volume−Initial Vehicle Treated Tumor Volume))*100.

### Example 1

This example was conducted according to the study procedure described above. The PRMT5 inhibitor was MRTX1719 administered at 100 mg/kg once a day (QD). MRTX1719 used is as described herein.

The immune checkpoint antibody targeting mouse-Ctla-4 was dosed on Day 0 while the antibody targeting mouse-Pd-1 was dosed on Day 0, 3 and 6.

Results are provided in Table 1. The combination of MRTX1719 and immune checkpoint blockade led to greater antitumor activity compared to either treatment regimens in the Mtap^{DEL} syngeneic model.

TABLE 1

| | Monotherapy Treatment Groups | | | |
|---|---|---|---|---|
| | | | MRTX1719 | Combination |
| | Vehicle + | | (100 QD) + | MRTX1719 |
| | Mouse | Vehicle + | Mouse | (100 QD) + |
| | IgG2b + | aCTLA-4 + | IgG2B + | aCTLA-4 + |
| Study Day | Rat IgG2a | aPD-1 | Rat IgG2B | aPD-1 |
| −1 | 82 | 82 | 82 | 81 |
| 1 | 108 | 105 | 115 | 105 |
| 3 | 165 | 128 | 118 | 109 |
| 6 | 263 | 152 | 154 | 103 |
| 8 | 374 | 206 | 182 | 110 |
| 10 | 450 | 254 | 233 | 94 |
| 13 | 750 | 418 | 352 | 119 |
| 15 | 985 | 594 | 558 | 173 |
| 17 | 1455 | 852 | 858 | 250 |
| 20 | — | 1140 | 1010 | 357 |
| 22 | — | 1365 | 1171 | 455 |
| 24 | — | 1573 | 1312 | 646 |
| 27 | — | — | 1644 | 865 |
| 29 | — | — | — | 957 |
| 31 | — | — | — | 1082 |
| 34 | — | — | — | 1200 |
| 37 | — | — | — | 1212 |
| 41 | — | — | — | 1559 |
| % TGI (Day 17) | — | 43.9 | 43.5 | 87.7 |

It is understood that the examples and embodiments described herein are for illustrative purposes only and that various modifications or changes in light thereof will be suggested to persons skilled in the art and are to be incorporated within the spirit and purview of this application and scope of the appended claims. All publications, patents, and patent applications cited herein are hereby incorporated herein by reference for all purposes.

This listing of the claims will replace all prior versions of claims in the application:

1. A method for treating cancer in a subject, the method comprising:
administering to the subject a therapeutically effective amount of one or more immuno-oncology checkpoint inhibitors and a therapeutically effective amount of a methylthioadenosine (MTA)-cooperative protein arginine N-methyl transferase 5 (PRMT5) inhibitor.

2. The method of claim 1, wherein the cancer comprises methylthioadenosine phosphorylase (MTAP) gene homozygous deletion.

3. The method of claim 1, wherein the cancer is a malignant peripheral nerve sheath tumors (MPNST), mesothelioma, pancreatic cancer, lung cancer, bladder cancer, head and neck cancer, esophageal cancer, diffuse large B cell lymphoma (DLBCL) cancer, stomach cancer, or melanoma.

4. The method of claim 1, wherein the cancer is lung cancer or pancreatic cancer.

5. The method of claim 1, wherein the immuno-oncology checkpoint inhibitor is selected from a CTLA-4 inhibitor, a PD-1 inhibitor, a PD-L1 inhibitor, and combinations thereof.

6. The method of claim 5, wherein the CTLA-4 inhibitor is selected from ipilimumab, tremelimumab.

7. The method of claim 5, wherein the PD-1 inhibitor is selected from pembrolizumab, cemiplimab, tislelizumab, nivolumab, and combinations thereof.

8. The method of claim 5, wherein the PD-L1 inhibitor is selected from avelumab, atezolizumab, durvalumab, BMS-936559, and combinations thereof.

9. The method of claim 1, wherein the PRMT5 inhibitor is: 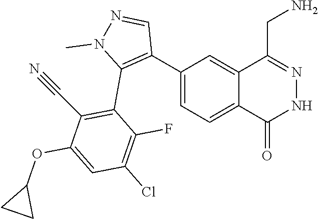 [SMILES: Cn1ncc(-c2ccc3c(=O)[nH]nc(CN)c3c2)c1-c1c(F)c(Cl)cc(OC2CC2)c1C#N]
or a pharmaceutically acceptable salt thereof.

10. The method of claim 1, wherein the PRMT5 inhibitor is: 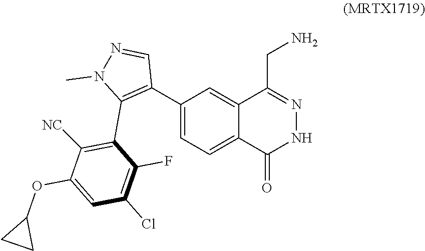
or a pharmaceutically acceptable salt thereof.

11. The method of claim 1, wherein the PRMT5 inhibitor is 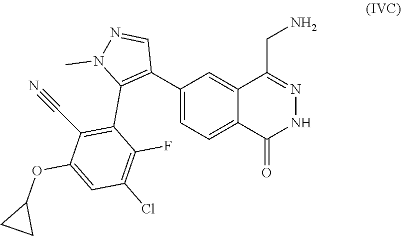 [SMILES: Cn1ncc(-c2ccc3c(=O)[nH]nc(CN)c3c2)c1-c1c(F)c(Cl)cc(OC2CC2)c1C#N]
or a pharmaceutically acceptable salt thereof, and the immuno-oncology checkpoint inhibitor is a CTLA4 inhibitor.

12. The method of claim 1, wherein the PRMT5 inhibitor is 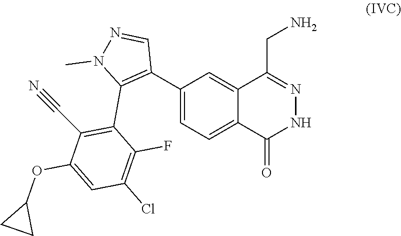 [SMILES: Cn1ncc(-c2ccc3c(=O)[nH]nc(CN)c3c2)c1-c1c(F)c(Cl)cc(OC2CC2)c1C#N]
or a pharmaceutically acceptable salt thereof, and the immuno-oncology checkpoint inhibitor is a PD-1 inhibitor.

13. The method of claim 1, wherein the PRMT5 inhibitor is 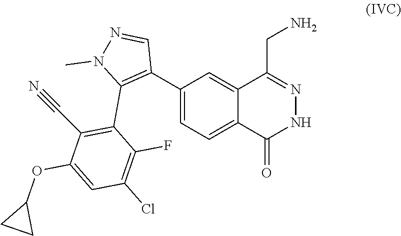 [SMILES: Cn1ncc(-c2ccc3c(=O)[nH]nc(CN)c3c2)c1-c1c(F)c(Cl)cc(OC2CC2)c1C#N]
or a pharmaceutically acceptable salt thereof, and the immuno-oncology checkpoint inhibitor is a PD-L1 inhibitor.

14. The method of claim 1, wherein the PRMT5 inhibitor is 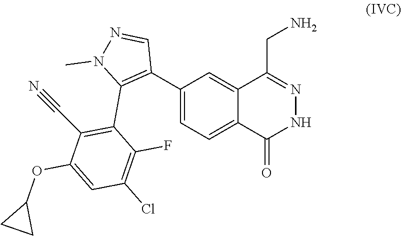 [SMILES: Cn1ncc(-c2ccc3c(=O)[nH]nc(CN)c3c2)c1-c1c(F)c(Cl)cc(OC2CC2)c1C#N]
or a pharmaceutically acceptable salt thereof, and the immuno-oncology checkpoint inhibitor comprises a CTLA4 inhibitor and a PD-1 inhibitor.

15. The method of claim 1, wherein the PRMT5 inhibitor is 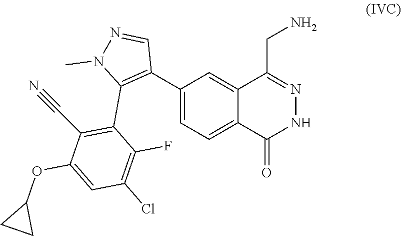 [SMILES: Cn1ncc(-c2ccc3c(=O)[nH]nc(CN)c3c2)c1-c1c(F)c(Cl)cc(OC2CC2)c1C#N]
or a pharmaceutically acceptable salt thereof, and the immuno-oncology checkpoint inhibitor comprises a CTLA4 inhibitor and a PD-L1 inhibitor.

16. The method of claim 1, wherein the PRMT5 inhibitor is MRTX1719 or a pharmaceutically acceptable salt thereof, and the immuno-oncology checkpoint inhibitor is a CTLA4 inhibitor.

17. The method of claim 1, wherein the PRMT5 inhibitor is MRTX1719 or a pharmaceutically acceptable salt thereof, and the immuno-oncology checkpoint inhibitor is a PD-1 inhibitor.

18. The method of claim 1, wherein the PRMT5 inhibitor is MRTX1719 or a pharmaceutically acceptable salt thereof, and the immuno-oncology checkpoint inhibitor is a PD-L1 inhibitor.

19. The method of claim 1, wherein the PRMT5 inhibitor is MRTX1719 or a pharmaceutically acceptable salt thereof, and the immuno-oncology checkpoint inhibitor comprises a CTLA4 inhibitor and a PD-1 inhibitor.

20. The method of claim 1, wherein the PRMT5 inhibitor is MRTX1719 or a pharmaceutically acceptable salt thereof, and the immuno-oncology checkpoint inhibitor comprises a CTLA4 inhibitor and a PD-L1 inhibitor.

21-30. (canceled)

31. The method of claim 1, wherein the subject previously received or completed a first-line chemotherapy.

32. The method of claim 1, wherein the subject did not previously received or complete a first-line chemotherapy.

33. The method of claim 31, wherein the first-line chemotherapy is platinum- and/or taxane-based chemotherapy.
